SEQUENCE LISTING (10 sequences)

SEQ ID NO: 1 - protein, 16 aa, synthetic construct
  REGION 1..16: note = CDR-L1 of AVE1642
RSSQSIVHSNVNTYLE

SEQ ID NO: 2 - protein, 7 aa, synthetic construct
  REGION 1..7: note = CDR-L2 of AVE1642
KVSNRFS

SEQ ID NO: 3 - protein, 9 aa, synthetic construct
  REGION 1..9: note = CDR-L3 of AVE1642
FQGSHVPPT

SEQ ID NO: 4 - protein, 150 aa, synthetic construct
  REGION 1..150: note = Light chain fragment of AVE1642
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKRTVAAPSVFIFPPSDEQLKSGTASVVCLLNNFYPREAK

SEQ ID NO: 5 - protein, 5 aa, synthetic construct
  REGION 1..5: note = CDR-H1 of AVE1642
SYWMH

SEQ ID NO: 6 - protein, 17 aa, synthetic construct
  REGION 1..17: note = CDR-H2 of AVE1642
EINPSNGRTNYNQKFQG

SEQ ID NO: 7 - protein, 15 aa, synthetic construct
  REGION 1..15: note = CDR-H3 of AVE1642
GRPDYYGSSKWYFDV

SEQ ID NO: 8 - protein, 150 aa, synthetic construct
  REGION 1..150: note = Heavy chain fragment of AVE1642
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALG

SEQ ID NO: 9 - protein, 113 aa, synthetic construct
  REGION 1..113: note = Light chain variable region of AVE1642
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 10 - protein, 124 aa, synthetic construct
  REGION 1..124: note = Heavy chain variable region of AVE1642
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

(19) United States
(12) Patent Application Publication
(10) Pub. No.: US 2025/0001019 A1
(43) Pub. Date: Jan. 2, 2025

(54) METHODS OF TREATING CANCER

(71) Applicant: Fusion Pharmaceuticals Inc., Hamilton (CA)

(72) Inventors: Eric S. Burak, Cambridge (CA); James O'Leary, Newton, MA (US); John Rhoden, Hingham, MA (US)

(21) Appl. No.: 18/703,995

(22) PCT Filed: Nov. 2, 2022
(86) PCT No.: PCT/US2022/079137

§ 371 Date: Apr. 23, 2024

Related U.S. Application Data
(60) Provisional application No. 63/274,802, filed on Nov. 2, 2021.

(51) Int. Cl. A61K 51/10; A61P 35/00

(52) U.S. Cl. CPC A61K 51/103; A61K 51/1096; A61P 35/00; A61K 2121/00

(57) ABSTRACT

Methods of treatment for conditions, e.g., cancer, using a cold IGF-1R-targeting molecule and a radioimmunoconjugate comprising a metal complex of a chelating moiety, a linker, and an IGF-1R targeting moiety.

### RELATED APPLICATIONS

The present application claims priority to U.S. Provisional Patent Application No. 63/274,802, filed Nov. 2, 2021, the entire contents of which are hereby incorporated by reference for all purposes.

### SEQUENCE LISTING

The instant application contains a Sequence Listing which has been submitted electronically in XML format and is hereby incorporated by reference in its entirety. Said XML file, created on Nov. 1, 2022, is named FPI_032_SL.xml and is 14 kilobytes in size.

### BACKGROUND

The insulin-like growth factor 1 receptor (IGF-1R) has been evaluated as a potential therapeutic target in the treatment of cancer. However, monotherapy trials with IGF-1R targeted antibodies or with IGF-1R specific tyrosine kinase inhibitors have, overall, been very disappointing in the clinical setting.

Thus, there remains a need for improved methods of treating cancer using therapeutics (e.g., cancer therapeutics) that can target IGF-1R.

### SUMMARY

The present disclosure relates to methods of treating cancer using radioimmunoconjugates that target IGF-1R (e.g., human IGF-1R). In certain embodiments, provided methods result in increased tumor uptake, reduced uptake in normal tissue(s), and/or result in decreased toxicity. Methods disclosed herein may, in some embodiments, allow a subject (e.g., a patient) to tolerate a higher radioactive dose than other methods using radioimmunoconjugates.

In one aspect, provided are methods of treating a patient having cancer, comprising (a) administering to a patient in need thereof an effective amount of a radioimmunoconjugate or pharmaceutically acceptable salt thereof, wherein the radioimmunoconjugate comprises the following structure:

A-L-B Formula I

wherein A is a metal complex of a chelating moiety, B is an IGF-1R targeting moiety, and L is a linker, and wherein the patient is being co-administered an IGF-1R targeting molecule.

In some embodiments, variable B is an antibody or an antigen-binding fragment thereof that is capable of binding to IGF-1R. In certain embodiments, the cold IGF-1R-targeting molecule is a cold IGF-1R antibody or IGF-1R binding fragment thereof.

In some embodiments, the cold IGF-1R antibody is pre-administered to the patient prior to the administration of a radioimmunoconjugate. In some embodiments, the cold IGF-1R antibody is co-administered to the patient together with the administration of a radioimmunoconjugate.

In some embodiments, the cold IGF-1R antibody is pre-administered at a dosage of 0.1 mg/kg to 10 mg/kg (e.g., 0.5 mg/kg to 3 mg/kg) based on the body weight of the patient.

In some embodiments, the radioimmunoconjugate is administered at a dosage of 10 kBq to 100 kBq/kg (e.g., 15 kBq to 80 kBq/kg) of body weight of said patient.

In some embodiments, the radioimmunoconjugate is administered with a cumulative exposure of 20 kBq to 300 kBq/kg (e.g., 20 kBq to 200 kBq/kg, 20 kBq to 150 kBq/kg, 20 kBq to 100 kBq/kg, or 35 kBq to 70 kBq/kg) of body weight of said patient.

In some embodiments, referring to Formula I, variable L has the structure of L¹-(L²)ₙ, as shown within Formula II:

A-L¹-(L²)ₙ-B Formula II
wherein
A is a metal complex of a chelating moiety;
B is an antibody or an antigen-binding fragment thereof that is capable of binding to IGF-1R;
L¹ is a bond, C═O, C═S, optionally substituted C₁-C₆ alkyl, optionally substituted C₁-C₆ heteroalkyl, or optionally substituted aryl or heteroaryl;
n is an integer between 1 and 5 (inclusive); and
each L², independently, has the structure:

—X¹-L³-Z¹—
wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and each R¹ is independently hydrogen, optionally substituted C₁-C₆ alkyl, optionally substituted C₁-C₆ heteroalkyl, or optionally substituted aryl or heteroaryl;
L³ is optionally substituted C₁-C₅₀ alkyl or optionally substituted C₁-C₅₀ heteroalkyl; and
Z¹ is —CH₂—, —C(O)—, —C(S)—, —OC(O)—#, —C(O)O-#, —NR²C(O)—#, —C(O)NR²-#, or —NR²—, wherein “#” indicates the attachment point to B, and each R² is independently hydrogen, optionally substituted C₁-C₆ alkyl, optionally substituted C₁-C₆ heteroalkyl, optionally substituted aryl, or optionally substituted heteroaryl.

In some embodiments, variable L³ comprises (CH₂CH₂O)₂₋₂₀ or (CH₂CH₂O)₂₋₂₀—C₁-C₆ alkyl.

In some embodiments, the chelating moiety is selected from the group consisting of DOTA (1,4,7,10-tetraazacyclododecane-1,4,7,10-tetraacetic acid), DOTMA (1R,4R,7R,10R)-α, α′, α″, α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DO3AM-acetic acid (2-(4,7,10-tris(2-amino-2-oxoethyl)-1,4,7,10-tetraazacyclododecan-1-yl)acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), NOTA (1,4,7-triazacyclononane-1,4,7-triacetic acid), and HP-DO3A (10-(2-hydroxypropyl)-1,4,7-tetraazacyclododecane-1,4,7-triacetic acid).

In some embodiments, the metal complex comprises a radionuclide selected from the group consisting of ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ^{99m}Tc, ¹⁰⁵Rh, ¹⁰⁹Ph, ¹¹¹In, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

In some embodiments, referring to Formula I, variable L has the structure of -L¹-(L²)ₙ-, as shown within Formula II:

A-L¹-(L²)ₙ-B Formula II

wherein:
A is a metal complex of DOTA;
B is an antibody or an antigen-binding fragment thereof that is capable of binding to IGF-1R;
L¹ is a bond or C₁-C₆ alkyl;
n is 1; and
L² has the structure:

—X¹-L³-Z¹—
wherein:
X¹ is —C(O)NR¹—*, “*” indicating the attachment point to L³, and R¹ is H or C₁-C₆ alkyl;
L³ is (CH₂CH₂O)ₘ(CH₂)_{w}, and m and w are independently an integral between 0 and 10 (inclusive) and at least one of m and w is not 0; and
Z¹ is —C(O)—.

In some embodiments, referring to Formula I, A-L- is a metal complex of a moiety selected from the group consisting of:

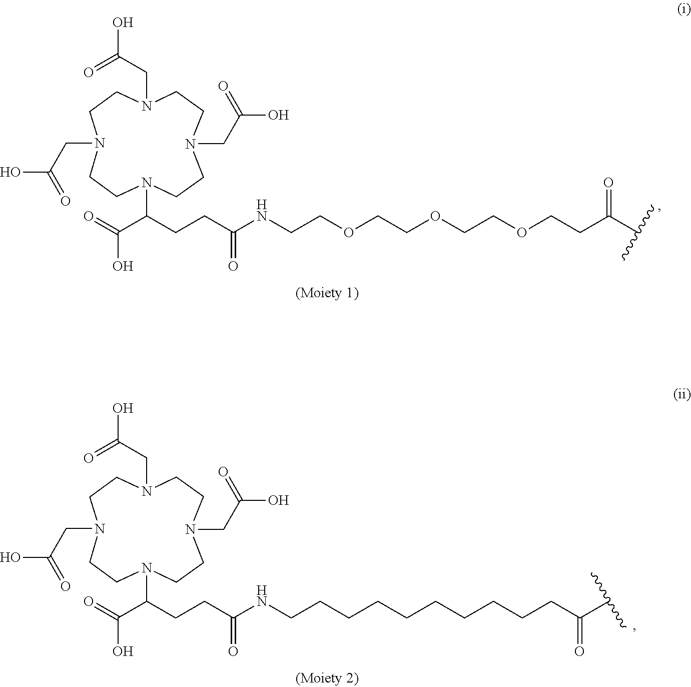 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1]

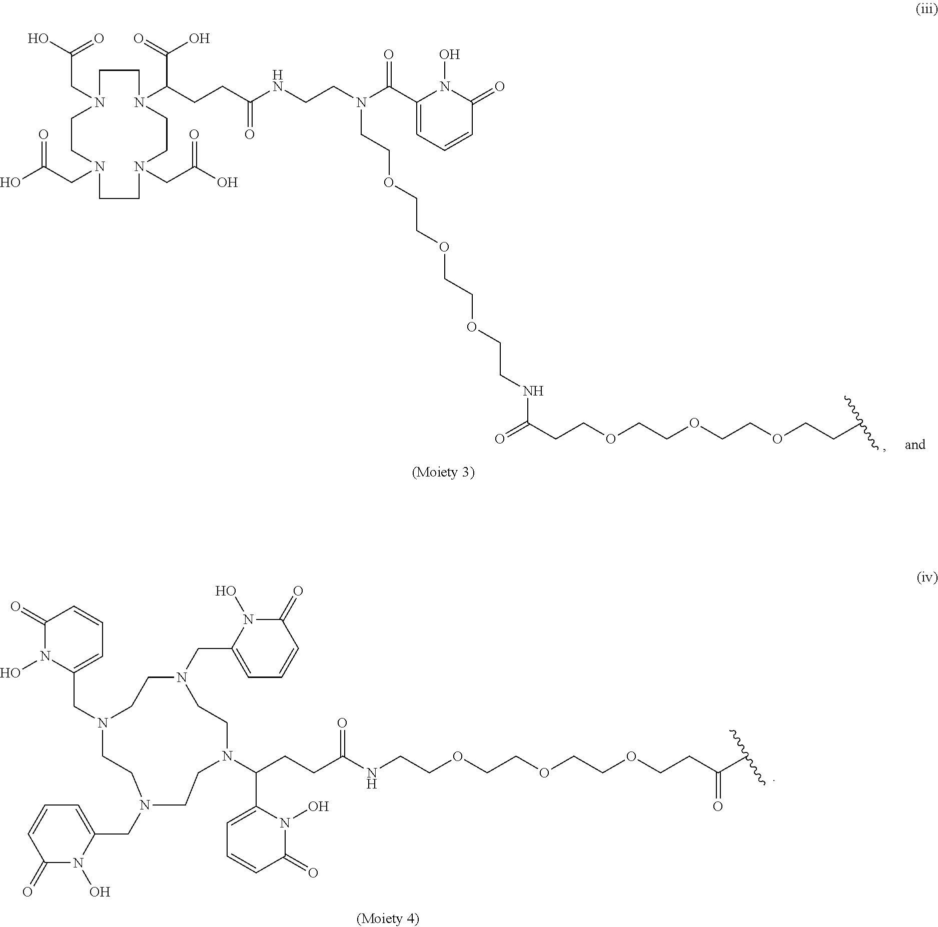 [SMILES: *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O ; *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

In some embodiments, A-L- is a metal complex of Moiety 1:

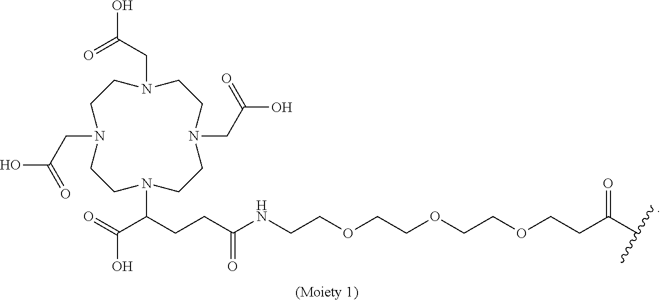 [SMILES: *C(=O)CCOCCOCCOCCNC(=O)CCC(C(=O)O)N1CCN(CC(=O)O)CCN(CC(=O)O)CCN(CC(=O)O)CC1]

In some embodiments, the radioimmunoconjugate comprises the following structure:

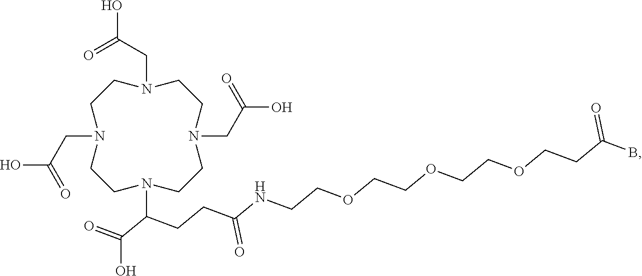

wherein B is an antibody or an antigen-binding fragment thereof that is capable of binding to IGF-1R.

In some embodiments, the metal complex comprises an alpha emitter. In certain embodiments, the alpha emitter is selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof. In certain embodiments, the metal complex comprises ²²⁵Ac or a progeny thereof.

In some embodiments, the radioimmunoconjugate comprises the following structure:

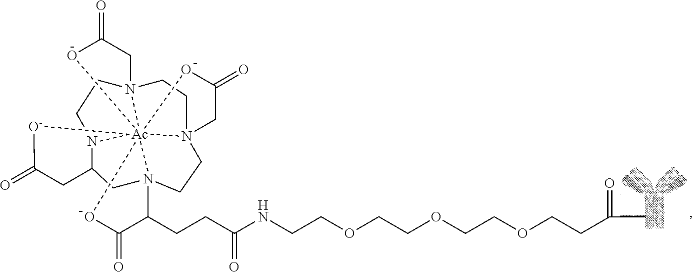

wherein [symbol] is IGF-1R antibody AVE1642.

In some embodiments, the method of the present disclosure features that the cold IGF-1R-targeting molecule is AVE1642 or an IGF-1R-binding fragment thereof.

In some embodiments, the method of the present disclosure can be used for treating cancer including, but not limited to, a solid tumor cancer.

In certain embodiments, the solid tumor cancer is selected from the group consisting of adrenocortical carcinoma, bladder cancer, breast cancer, cervical cancer, colorectal cancer, endometrial adenocarcinoma, Ewing's sarcoma, gallbladder carcinoma, glioma, head and neck cancer, liver cancer, lung cancer, neuroblastoma, neuroendocrine cancer, ovarian cancer, pancreatic cancer, prostate cancer, renal cell carcinoma, salivary adenoid cystic cancer, spermatocytic seminoma, and uveal melanoma.

### BRIEF DESCRIPTION OF THE DRAWINGS

FIG. 1A is a schematic depicting the general structure of bifunctional chelate comprising a chelate, a linker, and a cross-linking group.

FIG. 1B is a schematic depicting the general structure of a bifunctional conjugate comprising a chelate, a linker, and a targeting moiety.

FIGS. 1C-1D are schematics depicting the structures of [¹¹¹In]-DOTA-anti-IGF-1R and [²²⁵Ac]-DOTA-anti-IGF-1R, two exemplary radioimmunoconjugates disclosed herein.

FIG. 2 is a schematic depicting the synthesis of the bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B). Synthesis of Compound B is described in Example 2.

FIG. 3 is a schematic depicting the synthesis of the bifunctional chelate, 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C). Synthesis of Compound C is described in Example 3.

FIG. 4 is a schematic depicting the synthesis of IGF-1R-targeting radioimmunoconjugate [²²⁵Ac]-DOTA-anti-IGF-1R.

FIG. 5 shows a plot of pharmacokinetics of [¹¹¹In]-DOTA-anti-IGF-1R conjugate with and without cold IGF-1R antibody.

FIGS. 6A-6E show a plot of uptake of radioimmunoconjugate in various organs via imaging analysis.

FIGS. 7A-7E show a plot of radioimmunoconjugate tumor uptake relative to background in various organs via imaging analysis.

### DETAILED DESCRIPTION

Radioimmunoconjugates are designed to target a protein or receptor that is upregulated in a disease state to deliver a radioactive payload to damage and kill cells of interest (radioimmunotherapy). The process of delivering such a payload, via radioactive decay, produces an alpha, beta, or gamma particle or Auger electron that can cause direct effects to DNA (such as single or double stranded DNA breaks) or indirect effects such as by-stander or crossfire effects.

Radioimmunoconjugates typically contain a biological targeting moiety (e.g., an antibody or antigen binding fragment thereof that is capable of specifically binding to human IGF-1R), a radioisotope, and a molecule that links the two. Conjugates are formed when a bifunctional chelate is appended to the biological targeting molecule so that structural alterations are minimal while maintaining target affinity. Once radiolabelled, the final radioimmunoconjugate is formed.

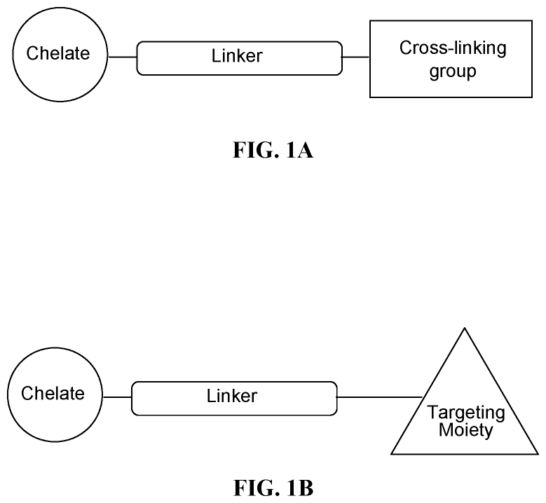

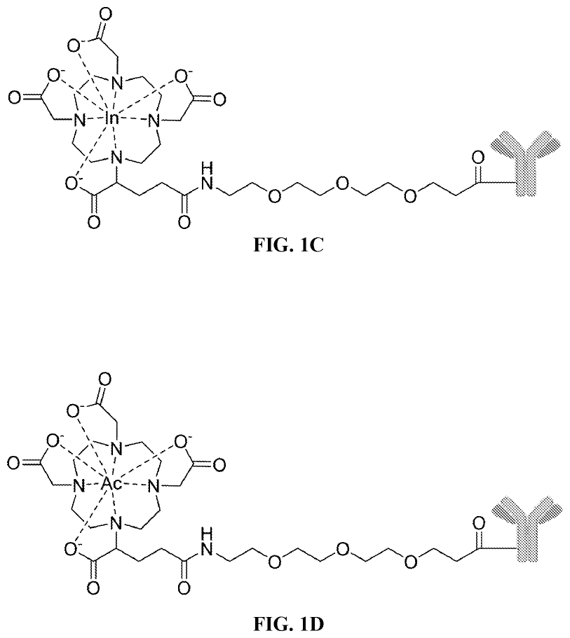

Bifunctional chelates structurally contain a chelate, a linker, and a cross-linking group (FIG. 1A). When developing new bifunctional chelates, most efforts focus around the chelating portion of the molecule. Several examples of bifunctional chelates have been described with various cyclic and acyclic structures conjugated to a targeted moiety. [Bioconjugate Chem. 2000, 11, 510-519; Bioconjugate Chem. 2012, 23, 1029-1039; Mol Imaging Biol. 2011, 13, 215-221, Bioconjugate Chem. 2002, 13, 110-115.]

One of the key factors of developing safe and effective radioimmunoconjugates is maximizing efficacy while minimizing off-target toxicity in normal tissue. While this statement is one of the core tenets of developing new drugs, the application to radioimmunotherapeutics presents new challenges. Radioimmunoconjugates do not need to block a receptor, as needed with a therapeutic antibody, or release the cytotoxic payload intracellularly, as required with an antibody drug conjugate, in order to have therapeutic efficacy. However, the emission of the toxic particle is an event that occurs as a result of first-order (radioactive) decay and can occur at random anywhere inside the body after administration. Once the emission occurs, damage could occur to surrounding cells within the range of the emission leading to the potential of off-target toxicity. Therefore, limiting exposure of these emissions to normal tissue is the key to developing new drugs.

One potential method for reducing off-target exposure is to remove the radioactivity more effectively from the body (e.g., from normal tissue in the body). One mechanism is to increase the rate of clearance of the biological targeting agent. This approach likely requires identifying ways to shorten the half-life of the biological targeting agent, which is not well described for biological targeting agents. Regardless of the mechanism, increasing drug clearance will also negatively impact the pharmacodynamics/efficacy in that the more rapid removal of drug from the body will lower the effective concentration at the site of action, which, in turn, would require a higher total dose and would not achieve the desired results of reducing total radioactive dose to normal tissue.

Other efforts have focused on accelerating the metabolism of the portion of the molecule that contains the radioactive moiety. To this end, some efforts have been made to increase the rate of cleavage of the radioactivity from the biological targeting agents using what have been termed “cleavable linkers”. Cleavable linkers, however, have been taken on different meaning as it relates to radioimmunoconjugates. Cornelissen, et al. has described cleavable linkers as those by which the bifunctional chelate attaches to the biologic targeting agent through a reduced cysteine, whereas others have described the use of enzyme-cleavable systems that require the co-administration of the radioimmunoconjugate with a cleaving agent/enzyme to release [Mol Cancer Ther. 2013, 12(11), 2472-2482; Methods Mol Biol. 2009, 539, 191-211; Bioconjug Chem. 2003, 14(5), 927-33]. These methods either change the nature of the biological targeting moiety, in the case of the cysteine linkage, or are not practical from a drug development perspective (enzyme cleavable systems) since, in the case of the citations provided, require the administration of two agents.

The present disclosure provides, among other things, methods of treating cancer using radioimmunoconjugates that, in various embodiments, result in increased tumor uptake, reduced uptake in normal tissue(s), and/or result in decreased toxicity. Methods disclosed herein may, in some embodiments, allow a subject (e.g., a patient) to tolerate a higher radioactive dose than other methods using radioimmunoconjugates.

One of the unique features of this invention includes dosing regimen of a radiopharmaceutical (imaging or therapeutic agent), e.g., a radioimmunoconjugate, in combination with a cold antibody, i.e., non-radioactive antibody, against the same target. The administration of non-radioactive antibody slows the clearance of the radiopharmaceutical and alters its biodistribution and kinetics. These effects result in increased systemic exposure to the radiopharmaceutical and favorable shifts in the lesion:normal organ radiation absorbed dose estimates based upon patient imaging and dosimetry, allowing patients to receive lower doses of the radiopharmaceutical while increasing radiation absorbed dose to the lesions.

The cumulative quantity of a radiopharmaceutical which can be administered is limited based upon normal organ limits (e.g., International Commission on Radiological Protection or ICRP, 2012 ICRP Statement on Tissue Reactions/Early and Late Effects of Radiation in Normal Tissues and Organs—Threshold Doses for Tissue Reactions in a Radiation Protection Context. ICRP Publication 118. Ann. ICRP 41(1/2)). The addition of non-radioactive antibody to the dosing regimen improves the tumor:normal organ uptake ratio, allowing for more cumulative radiation to be delivered to the tumor within the normal organ limit. It is expected that the delivery of higher amounts of radioactivity to the tumor may improve anti-tumor efficacy.

### Definitions

As used herein, the term “about” or “approximately,” when used in reference to a quantitative value, includes the recited quantitative value itself, unless specifically stated otherwise. As used herein, the term “about” or “approximately” refers to a ±10% variation from the recited quantitative value unless otherwise indicated or inferred from the context.

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains.

As used herein, “antigen-binding fragment” refers to a portion of an antibody that retains the binding characteristics of the parent antibody.

As used herein, the term “bind” or “binding” of a targeting moiety means an at least temporary interaction or association with or to a target molecule, e.g., to human IGF-1R, as described herein.

The term “bifunctional chelate,” as used herein, refers to a compound that comprises a chelate, a linker, and a cross-linking group. See, e.g., FIG. 1A. A “cross-linking group” is a reactive group that is capable of joining two or more molecules, e.g., joining a bifunctional chelate and a targeting moiety, by a covalent bond.

The term “bifunctional conjugate,” as used herein, refers to a compound that comprises a chelate or metal complex thereof, a linker, and a targeting moiety, e.g., an antibody or antigen-binding fragment thereof. See, e.g., Formula I or FIG. 1B.

The term “cancer” refers to any cancer caused by the proliferation of malignant neoplastic cells, such as tumors, neoplasms, carcinomas, sarcomas, leukemias, and lymphomas. A “solid tumor cancer” is a cancer comprising an abnormal mass of tissue, e.g., sarcomas, carcinomas, and lymphomas.

As used herein, the phrases “co-administer,” “administer in combination,” or “combined administration” means that two or more agents are administered to a subject at the same time or within an interval such that there may be an overlap of an effect of each agent on the subject. Thus, two or more agents that are administered in combination need not be administered together, although they may be. For example, one agent may be pre-administered before the other agent. In some embodiments, the two or more agents are administered within 24 hours (e.g., 12, 6, 5, 4, 3, 2, or 1 hour(s) of one another, or within about 60, 30, 15, 10, 5, or 1 minute(s) of one another. In some embodiments, the two or more agents are administered together, e.g., in the same formulation or, e.g., in different formulations but at the same time.

As used herein, the term “cold,” when used to describe an agent (e.g., a targeting moiety, such as an antibody or antigen-binding fragment thereof) means that the agent is not radioactive, e.g., not labeled with a radionuclide. A “cold” agent may or may not be conjugated to another moiety or modified in some way, so long as the cold agent is not radioactive.

The term “chelate,” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains a targeting moiety, e.g., an antibody or antigen-binding fragment thereof.

As used herein, the term “compound,” is meant to include all stereoisomers, geometric isomers, and tautomers of the structures depicted.

The compounds recited or described herein can be asymmetric (e.g., having one or more stereocenters). All stereoisomers, such as enantiomers and diastereomers, are intended unless otherwise indicated. Compounds discussed in the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

As used herein, “detection agent” refers to a molecule or atom which is useful in diagnosing a disease by locating the cells containing the antigen. Various methods of labeling polypeptides with detection agents are known in the art. Examples of detection agents include, but are not limited to, radioisotopes and radionuclides, dyes (such as with the biotin-streptavidin complex), contrast agents, luminescent agents (e.g., fluorescein isothiocyanate or FITC, rhodamine, lanthanide phosphors, cyanine, and near IR dyes), and magnetic agents, such as gadolinium chelates.

As used herein, the term “radionuclide” refers to an atom capable of undergoing radioactive decay (e.g., ³H, ¹⁴C, ¹⁵N, ¹⁸F, ³⁵S, ⁴⁴Sc, ⁴⁷Sc, ⁵⁵Co, ⁶⁰Cu, ⁶¹Cu, ⁶²Cu, ⁶⁴Cu, ⁶⁷Cu, ⁷⁵Br, ⁷⁶Br, ⁷⁷Br, ⁸⁹Zr, ⁸⁶Y, ⁸⁷Y, ⁹⁰Y, ⁹⁷Ru, ⁹⁹Tc, ^{99m}Tc, ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb, ²¹²Bi, ²¹³Bi, ²²³Ra, ²²⁵Ac, ²²⁷Th, ²²⁹Th, ⁶⁶Ga, ⁶⁷Ga, ⁶⁸Ga, ⁸²Rb, ^{117m}Sn, ²⁰¹Tl). The terms radioactive nuclide, radioisotope, or radioactive isotope may also be used to describe a radionuclide. Radionuclides may be used as detection agents, as described herein. In some embodiments, the radionuclide may be an alpha-emitting radionuclide.

The term an “effective amount” of an agent (e.g., any of the foregoing conjugates), as used herein, is that amount sufficient to effect beneficial or desired results, such as clinical results, and, as such, an “effective amount” depends upon the context in which it is being applied. For example, in therapeutic applications, an “effective amount” may be an amount sufficient to cure or at least partially arrest the symptoms of the disorder and its complications, and/or to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. An effective amount may be administered by administering a single dose or multiple (e.g., at least two, at least three, at least four, at least five, or at least six) doses.

The term “immunoconjugate,” as used herein, refers to a conjugate that includes a targeting moiety, such as an antibody (or antigen-binding fragment thereof), nanobody, affibody, or a consensus sequence from Fibronectin type III domain. In some embodiments, the immunoconjugate comprises an average of at least 0.10 conjugates per targeting moiety (e.g., an average of at least 0.2, 0.3, 0.4, 0.5, 0.6, 0.7, 0.8, 0.9, 1, 2, 4, 5, or 8 conjugates per targeting moiety).

The term “radioconjugate,” as used herein, refers to any conjugate that includes a radioisotope or radionuclide, such as any of the radioisotopes or radionuclides described herein.

The term “radioimmunoconjugate,” as used herein, refers to any immunoconjugate that includes a radioisotope or radionuclide, such as any of the radioisotopes or radionuclides described herein. A radioimmunoconjugate provided in the present disclosure typically refers to a bifunctional conjugate that comprises a metal complex formed from a radioisotope or radionuclide.

The term “radioimmunotherapy,” as used herein, refers a method of using a radioimmunoconjugate to produce a therapeutic effect. In some embodiments, radioimmunotherapy may include administration of a radioimmunoconjugate to a subject in need thereof, wherein administration of the radioimmunoconjugate produces a therapeutic effect in the subject. In some embodiments, radioimmunotherapy may include administration of a radioimmunoconjugate to a cell, wherein administration of the radioimmunoconjugate kills the cell. Wherein radioimmunotherapy involves the selective killing of a cell, in some embodiments the cell is a cancer cell in a subject having cancer.

The term “pharmaceutical composition,” as used herein, represents a composition containing a radioimmunoconjugate described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “polypeptide,” as used herein, refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

By “subject” is meant a human or non-human animal (e.g., a mammal).

By “substantial identity” or “substantially identical” is meant a polypeptide sequence that has the same polypeptide sequence, respectively, as a reference sequence, or has a specified percentage of amino acid residues, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Sequence identity may be measured using sequence analysis software on the default setting (e.g., Sequence Analysis Software Package of the Genetics Computer Group, University of Wisconsin Biotechnology Center, 1710 University Avenue, Madison, WI 53705). Such software may match similar sequences by assigning degrees of homology to various substitutions, deletions, and other modifications.

As used herein, the term “targeting moiety” refers to any molecule or any part of a molecule that is capable of binding to a given target. The term, “IGF-1R targeting moiety” refers to a targeting moiety that is capable of binding to an IGF-1R molecule, e.g., a human IGF-1R.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### Methods of Treatment

In one aspect, provided are methods of treating cancer comprise a step of administering to a subject (e.g., a patient) in need thereof a pharmaceutical composition comprising an effective amount of a radioimmunoconjugate as described further herein (e.g., a radioimmunoconjugate comprising an IGF-1R-targeting moiety), and wherein the subject is being co-administered a cold IGF-1R-targeting molecule.

By “cold IGF-1R-targeting molecule” it is meant that the IGF-1R-targeting molecule is not radioactive, e.g., not labeled with a radionuclide. An “IGF-1R-targeting molecule” as used herein refers to a molecule comprising an IGF-1R-targeting moiety, e.g., any IGF-1R-targeting moiety as described herein. For example, in some embodiments, the cold IGF-1R-targeting molecule is an antibody or antigen-binding fragment thereof that is capable of binding to IGF-1R. In some embodiments, the radioimmunoconjugate and the cold IGF-1R-targeting molecule are capable of binding the same epitope on IGF-1R.

### Administration

By “co-administered,” it is meant that the radioimmunoconjugate and the cold IGF-1R-targeting molecule are administered to a subject at the same time or within an interval such that there may be an overlap of an effect of each agent in the subject. The radioimmunoconjugate and the cold IGF-1R-targeting molecule need not be administered together, although they may be. For example, one agent may be pre-administered before the other agent. For example, in the context of the presently disclosed methods, the cold IGF-1R-targeting molecule may be pre-administered before the radioimmunoconjugate. For example, in some embodiments, a radioimmunoconjugate and a cold IGF-1R-targeting moiety are administered within 24 hours (e.g., 12, 6, 5, 4, 3, 2, or 1 hour(s) of one another, or within about 60, 30, 15, 10, 5, or 1 minute(s) of one another. In some embodiments, the IGF-1R-targeting moiety together, e.g., in the same formulation or, e.g., in different formulations but at the same time.

By “pre-administered,” it is meant that the cold IGF-1R-targeting molecule is administered before the radioimmunoconjugate is administered. For example, in some embodiments, the IGF-1R-targeting molecule is administered less than 5 hours, less than 4 hours, less than 3 hours, less than 2 hours before, less than 1 hour before, or less than 30 minutes before the radioimmunoconjugate is administered.

In some embodiments, the cold IGF-1R-targeting molecule is pre-administered prior to the administration of the radioimmunoconjugate.

Radioimmunoconjugates and pharmaceutical compositions thereof disclosed herein may be administered by any of a variety of routes of administration, including systemic and local routes of administration.

Systemic routes of administration include parenteral routes and enteral routes. In some embodiments, radioimmunoconjugates or pharmaceutical compositions thereof are administered by a parenteral route, for example, intravenously, intraarterially, intraperitoneally, subcutaneously, or intradermally. In some embodiments, radioimmunoconjugates or pharmaceutical compositions thereof are administered intravenously. In some embodiments, radioimmunoconjugates or pharmaceutical compositions thereof are administered by an enteral route of administration, for example, trans-gastrointestinal, or orally.

Local routes of administration include, but are not limited to, peritumoral injections and intratumoral injections.

### Dosages

Radioimmunoconjugates or pharmaceutical compositions comprising the same can be administered for radiation treatment planning, diagnostic, and/or therapeutic treatments. When administered for radiation treatment planning or diagnostic purposes, the radioimmunoconjugate may be administered to a subject in a diagnostically effective dose and/or an amount effective to determine the therapeutically effective dose. In therapeutic applications, pharmaceutical compositions may be administered to a subject (e.g., a patient) already suffering from a condition (e.g., cancer) in an amount sufficient to cure or at least partially arrest the symptoms of the disorder and its complications. An amount adequate to accomplish this purpose is defined as a “therapeutically effective amount,” an amount of a compound sufficient to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. In some embodiments, a subject is administered a first dose of a radioimmunoconjugate or composition in an amount effective for radiation treatment planning, then administered a second dose or set of doses of the radioimmunoconjugate or composition in a therapeutically effective amount.

Effective amounts may depend on the severity of the disease or condition and other characteristics of the subject (e.g., weight). Therapeutically effective amounts of disclosed radioimmunoconjugates and compositions for subjects (e.g., mammals such as humans) can be determined by the ordinarily-skilled artisan with consideration of individual differences (e.g., differences in age, weight, and the condition of the subject.

In some embodiments, the subject (e.g., a patient) is pre-administered or co-administered a dosage of 0.1 to 10 mg/kg (e.g., 0.2 to 8 mg/kg, 0.3 to 7 mg/kg, 0.4 to 6 mg/kg, 0.5 to 5 mg/kg, 0.5 to 4 mg/kg, 0.5 to 3 mg/kg, 0.5 to 2 mg/kg, or 0.5 to 1 mg/kg) of cold IGF-1R-targeting molecule (e.g., an IGF-1R antibody). In some embodiments, the patient is pre-administered or co-administered about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.5 mg/kg, about 2.0 mg/kg, about 2.5 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, or about 10 mg/kg of a cold IGF-1R antibody. In some embodiments, the patient is pre-administered a dosage of 0.5 to 3 mg/kg (e.g., 0.5 mg/kg or 1.5 mg/kg) of cold IGF-1R-targeting molecule (e.g., an IGF-1R antibody).

In some embodiments, the radioimmunoconjugate is administered at a dosage of 10 kBq to 100 kBq/kg (e.g., 15 kBq to 80 kBq/kg, 20 kBq to 60 kBq/kg, 25 kBq to 50 kBq/kg, 30 kBq to 40 kBq/kg, 25 kBq to 40 kBq/kg, 20 kBq to 40 kBq/kg, or 15 kBq to 40 kBq/kg) of body weight of said patient. In some embodiments, the radioimmunoconjugate is administered at a dosage of 15 kBq to 40 kBq/kg (e.g., about 15 kBq/kg, about 20 kBq/kg, about 25 kBq/kg, about 30 kBq/kg, about 35 kBq/kg, about 40 kBq/kg) of body weight of said patient.

In some embodiments, the radioimmunoconjugate is administered with a cumulative exposure of 20 kBq to 300 kBq/kg (e.g., 20 kBq to 200 kBq/kg, 20 kBq to 150 kBq/kg, 20 kBq to 100 kBq/kg, 25 kBq to 50 kBq/kg, 30 kBq to 60 kBq/kg, 35 kBq to 70 kBq/kg, or 35 kBq to 80 kBq/kg) of body weight of said patient.

To practice the methods of this invention, either the radioimmunoconjugate targeting IGF-1R or the cold IGF-1R-targeting molecule can be administered in a single dose or multiple doses (e.g., twice, three times, or four times within any given treatment).

Single or multiple administrations of radioimmunoconjugates disclosed herein including an effective amount can be carried out with dose levels and pattern being selected by the treating physician. Dose and administration schedule can be determined and adjusted based on the severity of the disease or condition in the subject, which may be monitored throughout the course of treatment according to the methods commonly practiced by clinicians or those described herein. With respect to radioactive doses, by way of non-limiting example, in some embodiments, the subject (e.g., a patient) is pre-administered about 0.5 to 3 mg/kg (e.g., 0.5 mg/kg or 1.5 mg/kg) of a cold IGF-1R antibody, followed by administration of an IGF-1R-targeting radioimmunoconjugate at a dosage of about 15 kBq to 40 kBq/kg (e.g., 15 kBq/kg or 30 kBq/kg).

### Functional Outputs

In some embodiments, a subject who has been treated with a method disclosed herein exhibits one or more improved characteristics as measured relative to a reference level. As used herein, the term “reference level” is a level as determined by the use of a control method in an experimental animal model or clinical trial. In some embodiments, the reference level refers to a level observed in a subject administered the same radioimmunoconjugate (and in some embodiments, with the same dosing protocol, including radioactive dose) but without co-administration of a cold IGF-1R-targeting molecule.

In some embodiments, a subject who has been treated with a method disclosed herein exhibits increased tumor uptake of the radioimmunoconjugate relative to a reference level, e.g., at least 1.2 times greater, at least 1.5 times greater, at least 2.0 times greater, at least 2.5 times greater, or at least 3 times greater levels in a tumor than a reference level at 24 h after administration of the radioimmunoconjugate. In some embodiments, a subject who has been treated with a method disclosed herein exhibits at least 1.2 times greater, at least 1.5 times greater, at least 2.0 times greater, at least 2.5 times greater, or at least 3 times greater levels in a tumor than a reference level at 48 h after administration of the radioimmunoconjugate. In some embodiments, a subject who has been treated with a method disclosed herein exhibits at least 1.2 times greater, at least 1.5 times greater, at least 2.0 times greater, at least 2.5 times greater, or at least 3 times greater in a tumor than a reference level at 96 h after administration of the radioimmunoconjugate.

In some embodiments, the subject exhibits a % ID/g of greater than 10%, greater than 15%, or greater than 20% in a tumor at 24 h after administration of the radioimmunoconjugate. In some embodiments, the subject exhibits a % ID/g of greater than 10%, greater than 15%, greater than 20%, greater than 25%, greater than 30%, greater than 35%, greater than 40%, or greater than 45% in a tumor at 96 h after administration of the radioimmunoconjugate.

In some embodiments, a subject who has been treated with a method disclosed herein exhibits reduced uptake of the radioimmunoconjugate in one or more normal (non-tumor) tissues relative to a reference level, e.g., 90% or less, 85% or less, 80% or less, 75% or less, 70% or less, 65% or less, 60% or less, 65% or less, or 50% or less of a reference level in one or more normal tissues at 24 h after administration of the radioimmunoconjugate. In some embodiments, the subject exhibits 90% or less, 85% or less, 80% or less, 75% or less, 70% or less, 65% or less, 60% or less, 65% or less, or 50% or less of a reference level in one or more normal tissues at 48 h after administration of the radioimmunoconjugate. In some embodiments, the subject exhibits 90% or less, 85% or less, 80% or less, 75% or less, 70% or less, 65% or less, 60% or less, 65% or less, or 50% or less of a reference level in one or more normal tissues at 96 h after administration of the radioimmunoconjugate.

In some embodiments, the subject exhibits a % ID/g of less than 10% in an internal organ (e.g., intestines, kidneys, adrenals, liver, gall bladder, lungs, spleen, skin, and/or bladder) at 4 h after administration of the radioimmunoconjugate. In some embodiments, the subject exhibits a % ID/g of less than 10% in an internal organ (e.g., intestines, kidneys, adrenals, liver, gall bladder, lungs, spleen, skin, and/or bladder) at 24 h after administration of the radioimmunoconjugate. In some embodiments, the subject exhibits a % ID/g of less than 10% in an internal organ (e.g., intestines, kidneys, adrenals, liver, gall bladder, lungs, spleen, skin, and/or bladder) at 48 h after administration of the radioimmunoconjugate. In some embodiments, the subject exhibits a % ID/g of less than 10% in an internal organ (e.g., intestines, kidneys, adrenals, liver, gall bladder, lungs, spleen, skin, and/or bladder) at 96 h after administration of the radioimmunoconjugate.

In some embodiments, a subject who has been treated with a method disclosed herein exhibits reduced clearance of the radioimmunoconjugate from the blood relative to a reference level, e.g., as evidenced by a higher % ID/g in the blood.

In some embodiments, a subject who has been treated with a method disclosed herein exhibits at least 5-fold, at least 10-fold, at least 20-fold, or at least 30-fold greater levels of radioactivity in the blood than a reference level at 24 h after administration of the radioimmunoconjugate. In some embodiments, a subject who has been treated with a method disclosed herein exhibits at least 5-fold, at least 10-fold, at least 20-fold, at least 30-fold greater levels of radioactivity in the blood than a reference level at 48 h after administration of the radioimmunoconjugate. In some embodiments, a subject who has been treated with a method disclosed herein exhibits at least 5-fold, at least 10-fold, at least 20-fold, at least 30-fold greater levels of radioactivity in the blood than a reference level at 96 h after administration of the radioimmunoconjugate.

In some embodiments, the subject exhibits a % ID/g of greater than 10%, greater than 15%, greater than 20%, or greater than 25% in the blood at 24 h after administration of the radioimmunoconjugate. In some embodiments, the subject exhibits a % ID/g of greater than 10%, greater than 12.5%, greater than 15%, or greater than 17.5% in the blood at 48 h after administration of the radioimmunoconjugate. In some embodiments, the subject exhibits a % ID/g of greater than 10%, greater than 12.5%, or greater than 15% in the blood at 96 h after administration of the radioimmunoconjugate.

In some embodiments, a subject who has been treated with a method disclosed herein exhibits reduced excretion of the radioimmunoconjugate in urine relative to a reference level, e.g., as evidenced by a lower % ID/g in the urine.

In some embodiments, a subject who has been treated with a method disclosed herein exhibits less than 75%, less than 70%, less than 65%, less than 60%, less than 55%, less than 50%, less than 45%, less than 40%, less than 35%, less than 30%, or less than 25% of levels of radioactivity in the urine as compared to a reference level at 24 h after administration of the radioimmunoconjugate. In some embodiments, a subject who has been treated with a method disclosed herein exhibits less than 75%, less than 70%, less than 65%, less than 60%, less than 55%, less than 50%, less than 45%, less than 40%, less than 35%, less than 30%, or less than 25% of levels of radioactivity in the urine as compared to a reference level at 48 h after administration of the radioimmunoconjugate. In some embodiments, a subject who has been treated with a method disclosed herein exhibits less than 75%, less than 70%, less than 65%, less than 60%, less than 55%, less than 50%, less than 45%, less than 40%, less than 35%, less than 30%, or less than 25% of levels of radioactivity in the urine as compared to a reference level at 96 h after administration of the radioimmunoconjugate.

In some embodiments, the subject exhibits a % ID/g of less than 10%, less than 8%, or less than 6% in urine at 24 h after administration of the radioimmunoconjugate. In some embodiments, the subject exhibits a % ID/g of less than 10% in urine at 96 h after administration of the radioimmunoconjugate.

In some embodiments, a subject who has been treated with a method disclosed herein exhibits reduced toxicity as compared to a reference level. In some embodiments, toxicity is assessed based on one or more of clinical observations (e.g., severity and/or frequency of side effects), food consumption, body weight, ophthalmologic examination, hematology, clinical chemistry, urinalysis, and examination of biopsy tissue.

In some embodiments, use of a method as disclosed herein allows a subject to tolerate a higher radioactive dose than a method in which the subject is not pre-administered or co-administered a cold IGF-1R-targeting molecule.

### Subjects

In some disclosed methods, a therapy (e.g., comprising a therapeutic agent) is administered to a subject. In some embodiments, the subject is a patient.

In some embodiments, the subject has cancer or is at risk of developing cancer. For example, the subject may have been diagnosed with cancer. For example, the cancer may be a primary cancer or a metastatic cancer. Subjects may have any stage of cancer, e.g., stage I, stage II, stage III, or stage IV with or without lymph node involvement and with or without metastases. Provided radioimmunoconjugates and compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the cancer is a solid tumor cancer, e.g., a sarcoma or carcinoma.

In some embodiments, the solid tumor cancer is adrenocortical carcinoma, bladder cancer (e.g., urothelial carcinoma), breast cancer (e.g., triple-negative breast cancer or TNBC), cervical cancer, colorectal cancer, endometrial adenocarcinoma, Ewing's sarcoma, gallbladder carcinoma, glioma (e.g., glioblastoma mutiforme), head and neck cancer (e.g., head and neck squamous cell carcinoma or HNSCC), liver cancer, lung cancer (e.g., small cell lung cancer or non-small cell lung cancer, or adenocarcinoma of the lung), neuroblastoma, neuroendocrine cancer, ovarian cancer, pancreatic cancer (e.g., pancreatic exocrine carcinoma), prostate cancer, renal cell carcinoma, salivary adenoid cystic cancer, spermatocytic seminoma, or uveal melanoma.

In some embodiments, the cancer is selected from the group consisting of bladder cancer, breast cancer, head and neck cancer, liver cancer, and lung cancer. In some embodiments, the cancer is bladder cancer. In some embodiments, the cancer is head and neck cancer. In some embodiments, the cancer is liver cancer. In some embodiments, the cancer is an unresectable or metastatic solid tumor that is a microsatellite instability-high (MSI-H) or a mismatch repair deficient (dMMR) solid tumor.

### Radioimmunoconjugates

Radioimmunoconjugates used in accordance with methods disclosed herein generally have the structure of Formula I:

A-L-B Formula I

wherein A is a chelating moiety or metal complex thereof, B is an IGF-1R targeting moiety, and L is a linker.

In some embodiments, the radioimmunoconjugate has or comprises the structure shown below:

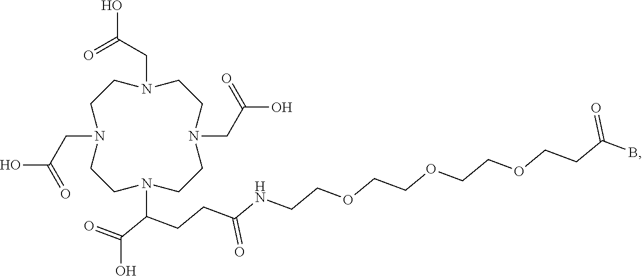

wherein B is the IGF-1R targeting moiety.

In some embodiments, A-L- is a metal complex of a moiety selected from the group consisting of

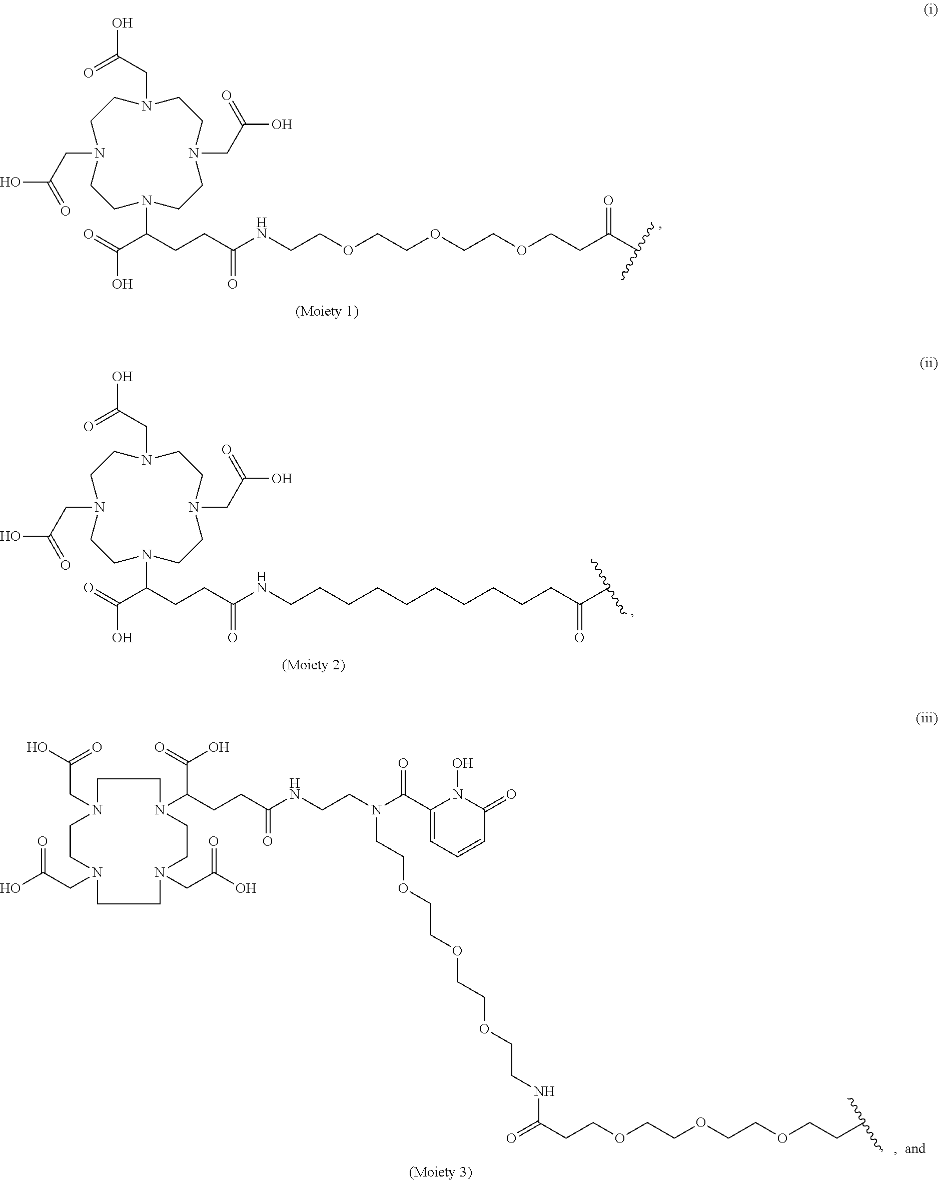 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCCN(CC(=O)O)CCN(CC(=O)O)CCCN(CC(=O)O)CC1)C(=O)c1cccc(=O)n1O]

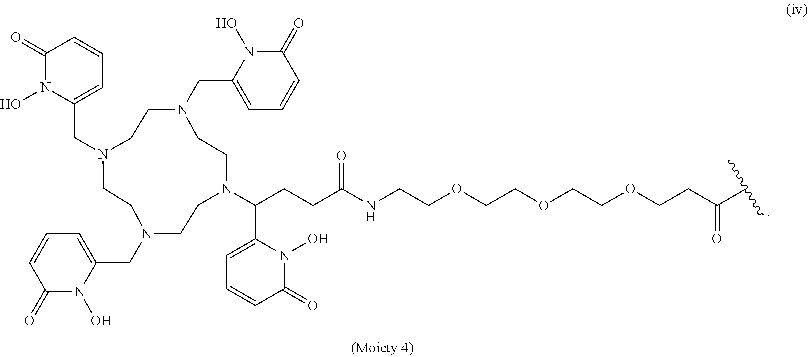 [SMILES: *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

In some embodiments, as further described herein, the radioimmunoconjugate comprises a chelating moiety or metal complex thereof, which metal complex may comprise a radionuclide. In some such radioimmunoconjugates, the average ratio or median ratio of the chelating moiety to the IGF-1R targeting moiety is eight or less, seven or less, six or less, five or less, four or less, three or less, two or less, or about one. In some radioimmunoconjugates, the average ratio or median ratio of the chelating moiety to the IGF-1R targeting moiety is about one.

In some embodiments, after the radioimmunoconjugate is administered to a patient, the proportion of radiation (of the total amount of radiation that is administered) that is excreted by the intestinal route, the renal route, or both is greater than the proportion of radiation excreted by a comparable patient that has been administered a reference radioimmunoconjugate. By “reference immunoconjugate” it is meant a known radioimmunoconjugate that differs from a radioimmunoconjugate described herein at least by (1) having a different linker; (2) having a targeting moiety of a different size and/or (3) lacking a targeting moiety. In some embodiments, the reference radioimmunoconjugate is selected from the group consisting of [⁹⁰Y]-ibritumomab tiuxetan (Zevalin (⁹⁰Y)) and [¹¹¹In]-ibritumomab tiuxetan (Zevalin (¹¹¹In)).

In some embodiments, the proportion of radiation excreted by a given route or set of routes) is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater than the proportion of radiation excreted by the same route(s) by a comparable patient that has been administered a reference radioimmunoconjugate. In some embodiments, the proportion of radiation excreted is at least 1.5-fold, at least 2-fold, at least 2.5-fold, at least 3-fold, at least 3.5 fold, at least 4-fold, at least 4.5 fold, at least 5 fold, at least 6-fold, at least 7-fold, at least 8-fold, at least 9-fold, or at least 10-fold greater than proportion of radiation excreted by a comparable patient that has been administered a reference radioimmunoconjugate. The extent of excretion can be measured by methods known in the art, e.g., by measuring radioactivity in urine and/or feces and/or by measuring total body radioactivity over a period time. See also, e.g., International Patent Publication WO 2018/024869.

In some embodiments, the extent of excretion is measured at a time period of at least or about 12 hours after administration, at least or about 24 hours after administration, at least or about 2 days after administration, at least or about 3 days after administration, at least or about 4 days after administration, at least or about 5 days after administration, at least or about 6 days after administration, or at least or about 7 days, after administration.

In some embodiments, after a radioimmunoconjugate has been administered to a patient, the radioimmunoconjugate exhibits decreased off-target binding effects (e.g., toxicities) as compared to a reference conjugate (e.g., a reference immunoconjugate such as a reference radioimmunoconjugate). In some embodiments, this decreased off-target binding effect is a feature of a radioimmunoconjugate that also exhibits a greater excretion rate as described herein.

### Targeting Moieties

Targeting moieties include any molecule or any part of a molecule that is capable of binding to a given target, e.g., IGF-1R. In some embodiments, the targeting moiety comprises a protein or polypeptide. In some embodiments, the targeting moiety is selected from the group consisting of antibodies or antigen binding fragments thereof, nanobodies, affibodies, and consensus sequences from Fibronectin type III domains (e.g., Centyrins or Adnectins). In some embodiments, a moiety is both a targeting and a therapeutic moiety, i.e., the moiety is capable of binding to a given target and also confers a therapeutic benefit. In some embodiments, the targeting moiety comprises a small molecule.

### Antibodies and Antigen-Binding Moieties

Antibodies typically comprise two identical light polypeptide chains and two identical heavy polypeptide chains linked together by disulfide bonds. The first domain located at the amino terminus of each chain is variable in amino acid sequence, providing the antibody-binding specificities of each individual antibody. These are known as variable heavy (VH) and variable light (VL) regions. The other domains of each chain are relatively invariant in amino acid sequence and are known as constant heavy (CH) and constant light (CL) regions. Light chains typically comprise one variable region (VL) and one constant region (CL). An IgG heavy chain includes a variable region (VH), a first constant region (CH1), a hinge region, a second constant region (CH2), and a third constant region (CH3). In IgE and IgM antibodies, the heavy chain includes an additional constant region (CH4).

Antibodies suitable for use with the present disclosure can include, for example, monoclonal antibodies, polyclonal antibodies, multispecific antibodies, human antibodies, humanized antibodies, camelid antibodies, chimeric antibodies, single-chain Fvs (scFv), disulfide-linked Fvs (sdFv), and anti-idiotypic (anti-Id) antibodies, and antigen-binding fragments of any of the above. In some embodiments, the antibody or antigen-binding fragment thereof is humanized. In some embodiments, the antibody or antigen-binding fragment thereof is chimeric. Antibodies can be of any type (e.g., IgG, IgE, IgM, IgD, IgA and IgY), class (e.g., IgG1, IgG2, IgG3, IgG4, IgA1 and IgA2) or subclass.

The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)2 fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

Antibodies or antigen-binding fragments described herein can be produced by any method known in the art for the synthesis of antibodies (See, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using the methods described in, e.g., Morrison, 1985, Science 229:1202, and humanized antibodies by methods described in, e.g., U.S. Pat. No. 6,180,370.

Additional antibodies described herein are bispecific antibodies and multivalent antibodies, as described in, e.g., Segal et al., J. Immunol. Methods 248:1-6 (2001); and Tutt et al., J. Immunol. 147: 60 (1991), or any of the molecules described herein.

“Avimer” relates to a multimeric binding protein or peptide engineered using, for example, in vitro exon shuffling and phage display. Multiple binding domains are linked, resulting in greater affinity and specificity compared to single epitope immunoglobin domains.

“Nanobodies” are antibody fragments consisting of a single monomeric variable antibody domain. Nanobodies may also be referred to as single-domain antibodies. Like antibodies, nanobodies are capable of binding selectively to a specific antigen. Nanobodies may be heavy-chain variable domains or light chain domains. Nanobodies may occur naturally or be the product of biological engineering. Nanobodies may be biologically engineered by site-directed mutagenesis or mutagenic screening (e.g., phage display, yeast display, bacterial display, mRNA display, ribosome display). “Affibodies” are polypeptides or proteins engineered to bind to a specific antigen. As such, affibodies may be considered to mimic certain functions of antibodies.

Affibodies may be engineered variants of the B-domain in the immunoglobulin-binding region of staphylococcal protein A. Affibodies may be engineered variants of the Z-domain, a B-domain that has lower affinity for the Fab region. Affibodies may be biologically engineered by site-directed mutagenesis or mutagenic screening (e.g., phage display, yeast display, bacterial display, mRNA display, ribosome display).

Affibody molecules showing specific binding to a variety of different proteins (e.g. insulin, fibrinogen, transferrin, tumor necrosis factor-α, IL-8, gp120, CD28, human serum albumin, IgA, IgE, IgM, HER2 and EGFR) have been generated, demonstrating affinities (Kd) in the M to pM range. “Diabodies” are antibody fragments with two antigen-binding sites that may be bivalent or bispecific. See for example Hudson et al., (2003). Single-chain antibodies are antibody fragments comprising all or a portion of the heavy chain variable domain or all, or a portion of the light chain variable domain of an antibody. Antibody fragments can be made by various techniques including but not limited to proteolytic digestion of an intact antibody as well as production by recombinant hosts (e.g., E. coli or phage) as described herein.

In certain embodiments, the antibody or antigen-binding fragment thereof is a multispecific, e.g. bispecific. Multispecific antibodies (or antigen-binding fragments thereof) include monoclonal antibodies (or antigen-binding fragments thereof) that have binding specificities for at least two different sites.

In certain embodiments, amino acid sequence variants of antibodies or antigen-binding fragments thereof are contemplated; e.g., variants that are capable of binding to human IGF-1R. For example, it may be desirable to improve the binding affinity and/or other biological properties of the antibody or antigen-binding fragment thereof. Amino acid sequence variants of an antibody or antigen-binding fragment thereof may be prepared by introducing appropriate modifications into the nucleotide sequence encoding the antibody or antigen-binding fragment thereof, or by peptide synthesis. Such modifications include, for example, deletions from and/or insertions into and/or substitutions of residues within the amino acid sequences of the antibody or antigen-binding fragment thereof. Any combination of deletion, insertion and substitution can be made to arrive at the final construct, provided that the final construct possesses desired characteristics, e.g. antigen binding.

In some embodiments, the antibody or antigen binding fragment thereof is an inhibitory antibody (also called “antagonistic antibody”) or antigen-binding fragment thereof, e.g., the antibody or antigen binding fragment thereof at least partially inhibits one or more functions of the target molecule (e.g., IGF-1R) as explained further herein.

In some embodiments, the antibody or antigen binding fragment thereof is an agonistic antibody (also known as stimulatory antibody).

Examples of antibodies, or antigen-binding fragments thereof capable of binding to IGF-1R include, but are not limited to, figitumumab, cixutumumab, dalotuzumab, ganitumab, AVE1642 (also known as humanized EM164 and huEM164), BIIB002, robatumumab, and teprotumumab, and antigen-binding fragments thereof. In some embodiments, the antibody, or an antigen binding fragment thereof is AVE1642 or an IGF-1R-binding fragment thereof.

In certain embodiments of the present disclosure, the antibody or antigen-binding fragment thereof comprises specific heavy chain complementarity determining regions CDR-H1, CDR-H2 and/or CDR-H3 as described herein. In some embodiments, the complementarity determining regions (CDRs) of the antibody or antigen-binding fragment thereof are flanked by framework regions. A heavy or light chain of an antibody or antigen-binding fragment thereof containing three CDRs typically contains four framework regions.

In some embodiments, the CDRs of the light chain variable region of AVE1642 have the sequences:

(CDR-L1)
SEQ ID NO: 1
RSSQSIVHSNVNTYLE
(CDR-L2)
SEQ ID NO: 2
KVSNRFS
(CDR-L3)
SEQ ID NO: 3
FQGSHVPPT

In some embodiments, the light chain variable region of AVE1642 has the sequence:

SEQ ID NO: 9
DVVMTQTPLSLPVSLGDPASISCRSSQSIVHSNVNTYLEWYLQKPG
QSPRLLIYKVSNRFSGVPDRFSGSGAGTDFTLRISRVEAEDLGIYY
CFQGSHVPPTFGGGTKLEIKR

In some embodiments, the light chain of AVE1642 comprises the sequence:

SEQ ID NO: 4
DVVMTQTPLSLPVSLGDPASISCRSSQSIVHSNVNTYLEWYLQKPG
QSPRLLIYKVSNRFSGVPDRFSGSGAGTDFTLRISRVEAEDLGIYY
CFQGSHVPPTFGGGTKLEIKRTVAAPSVFIFPPSDEQLKSGTASVV
CLLNNFYPREAK

In some embodiments, the CDRs of the heavy chain variable region of AVE1642 have the sequences:

(CDR-H1)
SEQ ID NO: 5
SYWMH
(CDR-H2)
SEQ ID NO: 6
EINPSNGRTNYNQKFQG
(CDR-H3)
SEQ ID NO: 7
GRPDYYGSSKWYFDV

In some embodiments, the heavy chain variable region of AVE1642 has the sequence:

SEQ ID NO: 10
QVQLVQSGAEVVKPGASVKLSCKASGYTFTSYWMHWVKQRPGQGLEW
IGEINPSNGRTNYNQKFQGKATLTVDKSSSTAYMQLSSLTSEDSAVY
YFARGRPDYYGSSKWYFDVWGQGTTVTVSS

In some embodiments, the heavy chain of AVE1642 comprises the sequence:

SEQ ID NO: 8
QVQLVQSGAEVVKPGASVKLSCKASGYTFTSYWMHWVKQRPGQGLEW
IGEINPSNGRTNYNQKFQGKATLTVDKSSSTAYMQLSSLTSEDSAVY
YFARGRPDYYGSSKWYFDVWGQGTTVTVSSASTKGPSVFPLAPSSKS
TSGGTAALG

In some embodiments, the antibody, or antibody-binding fragment thereof includes a light chain variable domain including at least one, two, or all three complementarity determining regions (CDRs) selected from:
(a) CDR-L1 including the amino acid sequence of SEQ ID NO: 1;
(b) CDR-L2 including the amino acid sequence of SEQ ID NO: 2; and
(c) CDR-L3 including the amino acid sequence of SEQ ID NO: 3.

In some embodiments, the antibody, or antibody-binding fragment thereof includes a heavy chain variable domain including at least one, two, or all three CDRs selected from:
(a) CDR-H1 including the amino acid sequence of SEQ ID NO: 5;
(b) CDR-H2 including the amino acid sequence of SEQ ID NO: 6; and
(c) CDR-H3 including the amino acid sequence of SEQ ID NO: 7.

In certain embodiments, the antibody, or antibody-binding fragment thereof includes a heavy chain variable domain and a light chain variable domain including at least one, two, three, four, five, or all six CDRs selected from:
(a) CDR-L1 including the amino acid sequence of SEQ ID NO: 1;
(b) CDR-L2 including the amino acid sequence of SEQ ID NO: 2;
(c) CDR-L3 including the amino acid sequence of SEQ ID NO: 3;
(d) CDR-H1 including the amino acid sequence of SEQ ID NO: 5;
(e) CDR-H2 including the amino acid sequence of SEQ ID NO: 6; and
(f) CDR-H3 including the amino acid sequence of SEQ ID NO: 7.

In some embodiments, the antibody, or antibody-binding fragment thereof features that the heavy chain variable domain includes the amino acid sequence of SEQ ID NO: 10.

In some embodiments, the antibody, or antibody-binding fragment thereof features that the light chain variable domain includes the amino acid sequence of SEQ ID NO: 9.

The antibody or antigen-binding fragment thereof may be any antibody or antigen-binding fragment thereof of natural and/or synthetic origin, e.g. an antibody of mammalian origin. In some embodiments, the constant domain, if present, is a human constant domain.

In some embodiments, the variable domain is a mammalian variable domain, e.g., a humanized or a human variable domain.

In some embodiments, antibodies used in accordance with this disclosure are monoclonal antibodies. In some embodiments, antibodies are recombinant murine antibodies, chimeric, humanized or fully human antibodies, multispecific antibodies (e.g., bispecific antibodies), or antigen-binding fragments thereof.

### Polypeptides

Polypeptides include, for example, any of a variety of hematologic agents (including, for instance, erythropoietin, blood-clotting factors, etc.), interferons, colony stimulating factors, antibodies, enzymes, and hormones. The identity of a particular polypeptide is not intended to limit the present disclosure, and any polypeptide of interest can be a polypeptide in the present methods.

A reference polypeptide described herein can include a target-binding domain that is capable of binding to a target of interest (e.g., is capable of binding to an antigen, e.g., IGF-1R). For example, a polypeptide, such as an antibody, can bind to a transmembrane polypeptide (e.g., receptor) or ligand (e.g., a growth factor).

### Modified Polypeptides

Polypeptides suitable for use with compositions and methods of the present disclosure may have a modified amino acid sequence. Modified polypeptides may be substantially identical to the corresponding reference polypeptide (e.g., the amino acid sequence of the modified polypeptide may have at least 50%, 60%, 70%, 75%, 80%, 85%, 90%, 95%, 96%, 97%, 98%, 99%, or 100% identity to the amino acid sequence of the reference polypeptide). In certain embodiments, the modification does not destroy significantly a desired biological activity (e.g., binding to IGF-1R). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified polypeptide may have or may optimize a characteristic of a polypeptide, such as in vivo stability, bioavailability, toxicity, immunological activity, immunological identity, and conjugation properties.

Modifications include those by natural processes, such as post-translational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from post-translational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy-terminus of a polypeptide herein can facilitate conjugation of these polypeptides by, e.g., disulfide bonding. For example, a polypeptide can be modified to include a single cysteine residue at the amino-terminus or a single cysteine residue at the carboxy-terminus. Amino acid substitutions can be conservative (i.e., wherein a residue is replaced by another of the same general type or group) or non-conservative (i.e., wherein a residue is replaced by an amino acid of another type). In addition, a naturally occurring amino acid can be substituted for a non-naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, N-protected amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 1. If such substitutions result in a change not desired, then other type of substitutions, denominated “exemplary substitutions” in Table 1, or as further described herein in reference to amino acid classes, are introduced and the products screened.

TABLE 1
Amino acid substitutions

| Original | | Conservative |
|---|---|---|
| residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Substantial modifications in function or immunological identity are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, and/or (c) the bulk of the side chain.

### Chelating Moiety or Metal Complex Thereof

### Chelating Moieties

Examples of suitable chelating moieties include, but are not limited to, DOTA (1,4,7,10-tetraazacyclododecane-1,4,7,10-tetraacetic acid), DOTMA (1R,4R,7R,10R)-α, α′, α″, α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DO3AM-acetic acid (2-(4,7,10-tris(2-amino-2-oxoethyl)-1,4,7,10-tetraazacyclododecan-1-yl)acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), NOTA (1,4,7-triazacyclononane-1,4,7-triacetic acid), and HP-DO3A (10-(2-hydroxypropyl)-1,4,7-tetraazacyclododecane-1,4,7-triacetic acid).

In some embodiments, the chelating moiety is DOTA.

In some embodiments, chelating moieties are useful as detection agents, and radioimmunoconjugates comprising such detectable chelating moieties can therefore be used as diagnostic or theranostic agents.

### Radioisotopes and Radionuclides

In some embodiments, the metal complex comprises a radionuclide. Examples of suitable radionuclides include, but are not limited to, ⁴⁴Sc, ⁴⁷Sc, ⁵⁵Co, ⁶⁰Cu, ⁶¹Cu, ⁶²Cu, ⁶⁴Cu, ⁶⁶Ga, ⁶⁷Ga, ⁶⁷Cu, ⁶⁸Ga, ⁷⁵Br, ⁷⁶Br, ⁷⁷Br, ⁸²Rb, ⁸⁹Zr, ⁸⁶Y, ⁸⁷Y, ⁹⁰Y, ⁹⁷Ru, ⁹⁹Tc, ^{99m}Tc, ¹⁰⁵Ph, ¹⁰⁹Pd, ¹¹¹In, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ^{117m}Sn, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

In some embodiments, the radionuclide is an alpha emitter, e.g., Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), or Terbium-149 (¹⁴⁹Tb), or a progeny thereof. In some embodiments, the alpha-emitter is Actinium-225 (²²⁵Ac), or a progeny thereof.

### Linker

In some embodiments, the linker is within the structure of Formula II as shown below:

A-L¹-(L²)ₙ-B Formula II

(A and B are as defined in Formula I.)

Thus, in some embodiments, the linker is -L¹-(L²)ₙ-, wherein:
L¹ is a bond, optionally substituted C₁-C₆ alkyl, optionally substituted C₁-C₆ heteroalkyl, or optionally substituted aryl or heteroaryl;
n is an integer between 1 and 5 (inclusive); and
each L², independently, has the structure:

—X¹-L³-Z¹— Formula III
wherein:
X¹ is-C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and each R¹ is independently hydrogen, optionally substituted C₁-C₆ alkyl (e.g., C₁-C₆ alkyl optionally substituted with oxo, heteroaryl, or a combination thereof), optionally substituted C₁-C₆ heteroalkyl, or optionally substituted aryl or heteroaryl;
L³ is optionally substituted C₁-C₅₀ alkyl or optionally substituted C₁-C₅₀ heteroalkyl (e.g., (CH₂CH₂O)₂₋₂₀); and
Z¹ is —CH₂—, —C(O)—, —C(S)—, —OC(O)—#, —C(O)O-#, —NR²C(O)—#, —C(O)NR²-#, or —NR²—, wherein “#” indicates the attachment point to B, and each R² is independently hydrogen, optionally substituted C₁-C₆ alkyl, optionally substituted C₁-C₆ heteroalkyl, optionally substituted aryl, or optionally substituted heteroaryl. In some embodiments, R² is pyrrolidine-2,5-dione.

In some embodiments, L¹ is substituted C₁-C₆ alkyl or substituted C₁-C₆ heteroalkyl, the substituent comprising a heteroaryl group (e.g., six-membered nitrogen-containing heteroaryl). In some embodiments, L¹ is C₁-C₆ alkyl. For example, L¹ is —CH₂CH₂—. In some embodiments, L¹ is a bond.

In some embodiments, X¹ is —C(O)NR¹—*, “*” indicating the attachment point to L³, and R¹ is H.

In some embodiments, L³ is optionally substituted C₁-C₅₀ alkyl (e.g., C₁-C₄₀ alkyl, C₁-C₃₀ alkyl, C₁-C₂₀ alkyl, C₂-C₁₈ alkyl, C₃-C₁₆ alkyl, C₄-C₁₄ alkyl, C₅-C₁₂ alkyl, C₆-C₁₀ alkyl, C₅-C₁₀ alkyl, or C₁₀ alkyl).

In some embodiments, L³ is optionally substituted C₁-C₅₀ heteroalkyl (e.g., C₁-C₄₀ heteroalkyl, C₁-C₃₀ heteroalkyl, C₁-C₂₀ heteroalkyl, C₂-C₁₈ heteroalkyl, C₃-C₁₆ heteroalkyl, C₄-C₁₄ heteroalkyl, C₅-C₁₂ heteroalkyl, C₆-C₁₀ heteroalkyl, C₅-C₁₀ heteroalkyl, C₄ heteroalkyl, C₆ heteroalkyl, C₈ heteroalkyl, C₁₀ heteroalkyl, C₁₂ heteroalkyl, C₁₆ heteroalkyl, C₂₀ heteroalkyl, or C₂₄ heteroalkyl).

In some embodiments, L³ is optionally substituted C₁-C₅₀ heteroalkyl comprising a polyethylene glycol (PEG) moiety comprising 1-20 oxyethylene (—O—CH₂—CH₂—) units, e.g., 2 oxyethylene units (PEG2), 3 oxyethylene units (PEG3), 4 oxyethylene units (PEG4), 5 oxyethylene units (PEG5), 6 oxyethylene units (PEG6), 7 oxyethylene units (PEG7), 8 oxyethylene units (PEG8), 9 oxyethylene units (PEG9), 10 oxyethylene units (PEG10), 12 oxyethylene units (PEG12), 14 oxyethylene units (PEG14), 16 oxyethylene units (PEG16), or 18 oxyethylene units (PEG18).

In certain embodiments, L³ is optionally substituted C₁₋₅₀ heteroalkyl comprising a polyethylene glycol (PEG) moiety comprising 1-20 oxyethylene (—O—CH₂—CH₂—) units or portions thereof. For example, L³ comprises PEG3 as shown below:

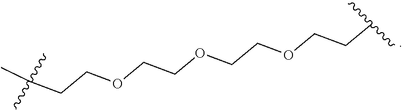 [SMILES: *CCOCCOCCOCC*]

In some embodiments, L³ is (CH₂CH₂O)ₘ(CH₂)_{w}, and m and w are each independently an integer between 0 and 10 (inclusive), and at least one of m and w is not 0.

In some embodiments, L³ is substituted C₁-C₅₀ alkyl or substituted C₁-C₅₀ heteroalkyl, the substituent comprising a heteroaryl group (e.g., six-membered nitrogen-containing heteroaryl).

In some embodiments, A is a macrocyclic chelating moiety comprising one or more heteroaryl groups (e.g., six-membered nitrogen-containing heteroaryl).

### Cross-Linking Groups

In some embodiments, radioimmunoconjugates are synthesized using bifunctional chelates that comprise a chelate, a linker, and a cross-linking group. Once the radioimmunoconjugate is formed, the cross-linking group may be absent from the radioimmunoconjugate.

In some embodiments, radioimmunoconjugates comprise a cross-linking group instead of or in addition to the targeting moiety (e.g., in some embodiments, B in Formula I comprises a cross-linking group).

A cross-linking group is a reactive group that is able to join two or more molecules by a covalent bond. Cross-linking groups may be used to attach the linker and chelating moiety to a therapeutic or targeting moiety. Cross-linking groups may also be used to attach the linker and chelating moiety to a target in vivo. In some embodiments, the cross-linking group is an amino-reactive, methionine reactive or thiol-reactive cross-linking group, or comprises a sortase recognition sequence. In some embodiments, the amino-reactive or thiol-reactive cross-linking group comprises an activated ester such as a hydroxysuccinimide ester, 2,3,5,6-tetrafluorophenol ester, 4-nitrophenol ester or an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine. In some embodiments, the sortase recognition sequence may comprise of a terminal glycine-glycine-glycine (GGG) and/or LPTXG amino acid sequence, where X is any amino acid. A person having ordinary skill in the art will understand that the use of cross-linking groups is not limited to the specific constructs disclosed herein, but rather may include other known cross-linking groups.

### Pharmaceutical Compositions

Pharmaceutical compositions comprising radioimmunoconjugates for use in methods disclosed herein can be formulated for use in a variety of drug delivery systems. One or more physiologically acceptable excipients or carriers can also be included in a pharmaceutical composition for proper formulation. Non-limiting examples of suitable formulations compatible for use with the present disclosure include those described in Remington's Pharmaceutical Sciences, Mack Publishing Company, Philadelphia, PA, 17th ed., 1985. For a brief review of methods for drug delivery, See, e.g., Langer (Science. 249:1527-1533, 1990).

Pharmaceutical compositions may be formulated for any of a variety of routes of administration discussed herein (See, e.g., the “Administration and Dosage” subsection herein), Sustained release administration is contemplated, by such means as depot injections or erodible implants or components. Thus, the present disclosure provides pharmaceutical compositions that include agents disclosed herein (e.g., radioimmunoconjugates) dissolved or suspended in an acceptable carrier, preferably an aqueous carrier, e.g., water, buffered water, saline, or PBS, among others. In some embodiments, pharmaceutical compositions contain pharmaceutically acceptable auxiliary substances to approximate physiological conditions, such as pH adjusting and buffering agents, tonicity adjusting agents, wetting agents, or detergents, among others. In some embodiments, pharmaceutical compositions are formulated for oral delivery and may optionally contain inert ingredients such as binders or fillers for the formulation of a unit dosage form, such as a tablet or a capsule. In some embodiments, pharmaceutical compositions are formulated for local administration and may optionally contain inert ingredients such as solvents or emulsifiers for the formulation of a cream, an ointment, a gel, a paste, or an eye drop.

In some embodiments, provided pharmaceutical compositions are sterilized by conventional sterilization techniques, e.g., may be sterile filtered. Resulting aqueous solutions may be packaged for use as is, or lyophilized. Lyophilized preparations can be, for example, combined with a sterile aqueous carrier prior to administration. The pH of preparations typically will be between 3 and 11, more preferably between 5 and 9 or between 6 and 8, and most preferably between 6 and 7, such as 6 to 6.5. Resulting compositions in solid form may be packaged, for example, in multiple single dose units, each containing a fixed amount of the above-mentioned agent or agents, such as in a sealed package of tablets or capsules. Pharmaceutical compositions in solid form can also be packaged in a container for a flexible quantity, such as in a squeezable tube designed for a topically applicable cream or ointment.

The following specific examples are to be construed as merely illustrative, and not limitative of the remainder of the disclosure in any way whatsoever.

### EXAMPLES

### Example 1. General Materials and Methods

Lutetium-177 can be obtained from Perkin Elmer as lutetium trichloride in a 0.05 N hydrochloric acid solution; indium-111, as a trichloride salt, can be obtained from Nordion; and actinium-225 can be obtained as actinium-225 trinitrate from Oak Ridge National Laboratories or actinium-225 trichloride from Canadian Nuclear Laboratories.

Analytical HPLC-MS can be performed using a Waters Acquity HPLC-MS system comprised of a Waters Acquity Binary Solvent Manager, a Waters Acquity Sample Manager (samples cooled to 10° C.), a Water Acquity Column Manager (column temperature 30° C.), a Waters Acquity Photodiode Array Detector (monitoring at 254 nm and 214 nm), a Waters Acquity TQD with electrospray ionization and a Waters Acquity BEH C18, 2.1×50 (1.7 μm) column. Preparative HPLC can be performed using a Waters HPLC system comprised of a Waters 1525 Binary HPLC pump, a Waters 2489 UV/Visible Detector (monitoring at 254 nm and 214 nm) and a Waters XBridge Prep phenyl or C18 19×100 mm (5 μm) column.

HPLC elution method 1: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; initial=90% A, 3-3.5 min=0% A, 4 min=90% A, 5 min=90% A.

HPLC elution method 2: Waters XBridge Prep Phenyl 19×100 mm (5 μm) column; mobile phase A: H₂O (0.1% v/v TFA); mobile phase B: acetonitrile (0.1% v/v TFA); flow rate: 10 mL/min; initial=80% A, 13 min=0% A.

HPLC elution method 3: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; initial=90% A, 8 min=0% A, 10 min=0% A, 11 min=90% A, 12 min=90% A.

HPLC elution method 4: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=80% A, 3 min=80% A, 13 min=20% A, 18 min=0% A.

HPLC elution method 5: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=90% A, 3 min=90% A, 13 min=0% A, 20 min=0% A.

HPLC elution method 6: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=75% A, 13 min=0% A, 15 min=0% A.

HPLC elution method 7: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=80% A, 12 min=0% A, 15 min=0% A.

HPLC elution method 8: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=90% A, 12 min=0% A, 15 min=0% A.

Analytical Size Exclusion Chromatography (SEC) can be performed using a Waters system comprised of a Waters 1525 Binary HPLC pump, a Waters 2489 UV/Visible Detector (monitoring at 280 nm), a Bioscan Flow Count radiodetector (FC-3300) and TOSOH TSKgel G3000SWxl, 7.8×300 mm column. The isocratic SEC method can have a flow rate of, e.g., mL/min, with a mobile phase of 0.1 M phosphate, 0.6 M NaCl, 0.025% sodium azide, pH=7.

MALDI-MS (positive ion) can be performed using a MALDI Bruker Ultraflextreme Spectrometer.

Radio thin-layer chromatography (radioTLC) can be performed with Bioscan AR-2000 Imaging Scanner, and can be carried out on iTLC-SG glass microfiber chromatography paper (Agilent Technologies, SGI0001) plates using citrate buffer (0.1 M, pH 5.5).

### Example 2. Synthesis of 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B)

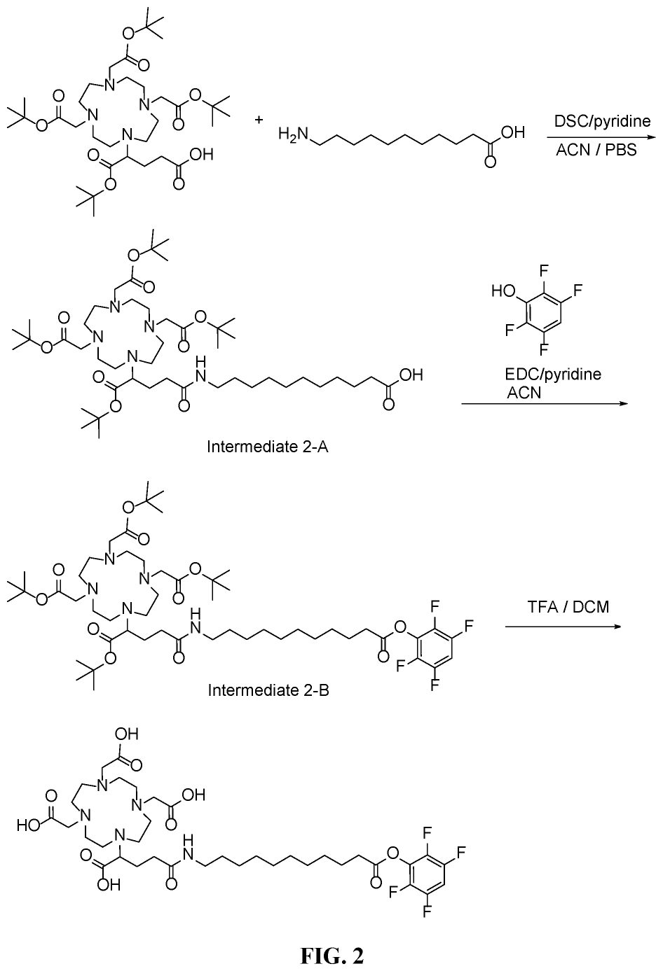

A bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B), can be synthesized according to the scheme provided in FIG. 2. To a solution of 5-(tert-butoxy)-5-oxo-4-(4,7,10-tris(2-(tert-butoxy)-2-oxoethyl)-1,4,7,10-tetraazacyclododecan-1-yl)pentanoic acid (DOTA-GA-(tBu)₄, 50 mg, 0.07 mmol) in ACN (2.0 mL), DSC (50 mg, 0.21 mmol) is added, followed by pyridine, (0.20 mL, 2.48 mmol). The reaction is stirred at room temperature for 1 hour. To the reaction mixture is added 11-aminoundecanoic acid, (70 mg, 0.36 mmol) followed by PBS solution (1.0 mL) at room temperature. The reaction is stirred for 72 hours at room temperature. The reaction mixture is filtered with a syringe filter and purified directly by Prep-HPLC using method 6 to yield Intermediate 2-A.

To a solution of Intermediate 2-A (40 mg, 0.03 mmol), TFP (90 mg, 0.54 mmol) and EDC (40 mg, 0.27 mmol) in ACN (1.0 mL) is added pyridine (0.05 mL, 50 mg, 0.62 mmol) at room temperature. The solution is stirred at room temperature for 24 hours. The reaction is purified directly by Prep-HPLC using method 7 to provide Intermediate 2-B as a wax after concentration using a Biotage V10 Rapid Evaporator.

Intermediate 2-B is dissolved in DCM/TFA (1.0 mL/2.0 mL) and allowed to stir at room temperature for 24 hours. The reaction is concentrated by air stream and purified directly by Prep-HPLC using method 8 to yield Compound B as a clear wax after concentration. An aliquot is analyzed by HPLC-MS elution method 3.

¹H NMR (600 MHz, DMSO-d6) δ 7.99-7.88 (m, 1H), 7.82 (t, J=5.5 Hz, 1H), 3.78 (broad s, 4H), 3.43 (broad s, 12H), 3.08 (broad s, 4H), 3.00 (m, 3H), 2.93 (broad s, 3H), 2.77 (t, J=7.2 Hz, 2H), 2.30 (broad s, 2H), 1.88 (broad s, 2H), 1.66 (p, J=7.3 Hz, 2H), 1.36 (m, 4H), 1.32-1.20 (m, 9H).

### Example 3. Synthesis of 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C)

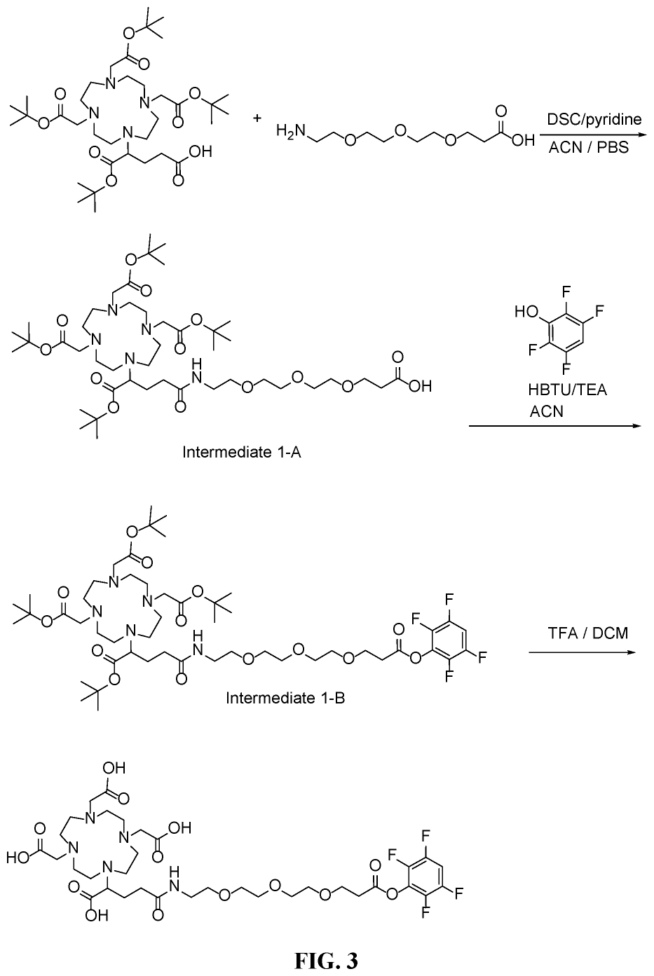

A bifunctional chelate, 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C), is synthesized according to the scheme provided in FIG. 3.

To a solution of 5-(tert-butoxy)-5-oxo-4-(4,7,10-tris(2-(tert-butoxy)-2-oxoethyl)-1,4,7,10-tetraazacyclododecan-1-yl)pentanoic acid (DOTA-GA(tBu)₄, 100 mg, 0.143 mmol) in ACN (8.0 mL) is added DSC (73 mg, 0.285 mmol) and pyridine (0.80 mL, 9.89 mmol). The reaction mixture is stirred for 90 min at ambient temperature. This solution is added to a semi-solution of amino-PEG3-acid (63 mg, 0.285 mmol in 1.2 mL of DMF) in a 100 mL round bottom flask. After 4 hours at ambient temperature, the reaction is worked up by concentrating to dryness under a stream of air. The crude material is purified by HPLC elution method 2 (dissolved the crude in 6 mL of 20% ACN/H₂O). The fractions containing product are pooled and concentrated under vacuum and then co-evaporated with ACN (3×2 mL).

To a vial containing Intermediate 1-A (82 mg, 60 μmol) is added ACN (2 mL), NEt₃ (50 μL, 360 μmol, 6 equiv.), HBTU (23 mg, 60 μmol, 1 equiv) and a TFP solution (50 mg, 300 μmol, 5 equiv., dissolved in 250 μL of ACN). The resulting clear solution is stirred at ambient temperature for 3 hours. The reaction is worked up by concentrating the solution to dryness under an air stream and is then diluted with ACN/H₂O (1:1, 3 mL total) and purified on preparative HPLC using elution method 4. Fractions containing product are pooled and concentrated under vacuum and then co-evaporated with ACN (3×2 mL). Intermediate 1-B is obtained as a clear residue.

To a vial containing Intermediate 1-B (67 mg, 64 μmol) is added DCM (2 mL) and TFA (2 mL). The resulting solution is stirred at ambient temperature for 16 hour. Additional, TFA (2 mL) is added, and the reaction is stirred at ambient temperature for 6 hours. The reaction is concentrated to dryness under an air stream, with the crude product being finally dissolved in ACN/H₂O (1 mL of 10% ACN/H₂O). The crude reaction solution is then purified by preparative HPLC using elution method 5. The fractions containing product are pooled, frozen and lyophilized. Compound C is obtained as a white solid. An aliquot is analyzed by HPLC-MS elution method 3.

¹H NMR (DMSO-d6, 600 MHz) δ 7.97-7.91 (m, 2H), 3.77 (t, 2H, J=6.0 Hz), 3.58-3.55 (m, 2H), 3.53-3.48 (m, 8H), 3.44-3.38 (m, 10H), 3.23-3.08 (m, 11H), 3.02 (t, 2H, J=6.0 Hz), 2.93 (broad s, 4H), 2.30 (broad s, 2H), 1.87 (broad s, 2H).

### Example 4. Synthesis of Radioimmunoconjugates Targeting IGF-1R

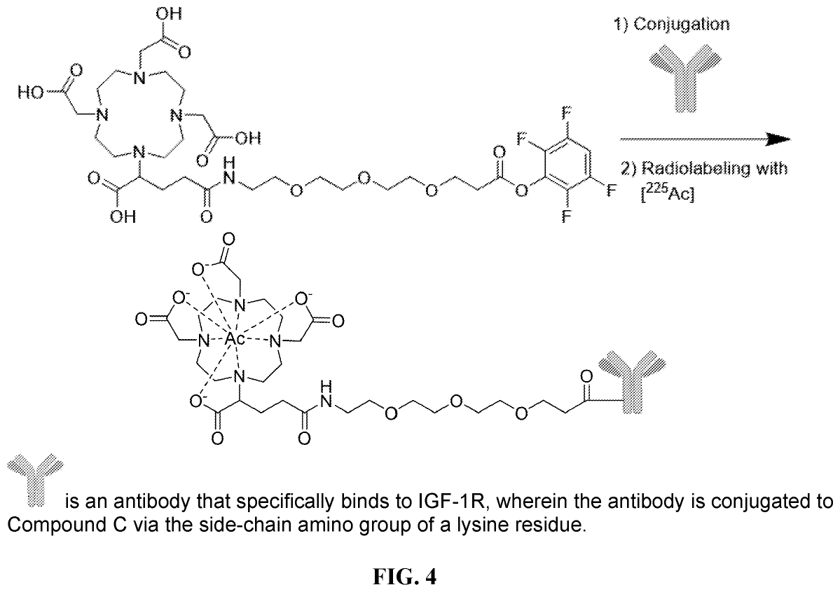

FIG. 4 shows the steps for synthesis of two radioimmunoconjugates targeting IGF-1R, namely, [¹¹¹In]-DOTA-anti-IGF-1R conjugate and [²²⁵Ac]-DOTA-anti-IGF-1R conjugate. The synthesis followed the protocols below.

### [¹¹¹In]-Compound C-Anti-IGF-1R Conjugate

Compound C (17.5 μmoles) was dissolved in sodium acetate buffer (1.32 mL, pH 6.5). An aliquot of Compound C solution (8 μL, 91 nmoles) was added to a solution containing the antibody IGF-1R (13.4 nmoles) in a bicarbonate buffer (pH 8.5). After 1 hour at ambient temperature, the resulting immunoconjugate was purified via a Sephadex G-50 resin packed column. The immunoconjugate Compound C-anti-IGF-1R was eluted from the column with acetate buffer (pH 6.5). MALDI-TOF-MS (positive ion): Compound C-anti-IGF-1R found m/z 152166 [M+H]+; IGF-1R found m/z 149724 [M+H]+.

As a typical reaction, In-111 (60 mCi, 215 μL) was added to a solution of Compound C-anti-IGF-1R (7 mg, 1.6 mL in acetate buffer (pH 6.5)) and ascorbic acid (100 μL, 0.2 Min acetate buffer (pH 6.5)). The radiolabeling reaction was incubated at 37° C. for 30 minutes. [¹¹¹In]-Compound C-anti-IGF-1R conjugate was purified via a Sephadex G-50 resin packed column eluted with acetate buffer (pH 6.5, 1 mM ascorbic acid).

### [²²⁵Ac]-Compound C-Anti-IGF-1R Conjugate

Compound C (1 μmole) is dissolved in a hydrochloric acid solution (0.001 M). An aliquot of Compound C solution (5 μL, 70 nmole) is added to a solution containing anti-IGF-1R antibody (1.8 nmoles) in a phosphate buffer (pH 8). After 3 hours at ambient temperature, the resulting immunoconjugate is purified via a Sephadex G-50 resin packed column. The immunoconjugate Compound C-anti-IGF-1R is eluted from the column with acetate buffer (pH 6.5). Identities of eluates can be confirmed by, e.g., MALDI-TOF. Ac-225 (15 μCi, 10 μL) is added to a solution of Compound C-anti-IGF-1R (300 μg in acetate buffer (pH 6.5). The radiolabeling reaction is incubated at 30° C. for 1 hour. The crude product, [²²⁵Ac]-Compound C-anti-IGF-1R, is purified via a Sephadex G-50 resin packed column eluted with acetate buffer.

### Example 5. Imaging and Pharmacokinetics of IGF-1R-Targeting Radioimmunoconjugates Involving Cold IGF-1R Antibody Pre-Administration

A study was performed to evaluate the imaging and pharmacokinetics in patients of receiving cold IGF-1R antibody pre-administration prior to administration of [¹¹¹In]-DOTA-anti-IGF-1R conjugate. The study followed the protocols below.

Five patients were evaluated in the cold antibody pre-administration study at two different dose levels of cold antibody. Patients were first imaged and samples were taken for pharmacokinetic analysis after receiving a fixed dose of 5 mCi [¹¹¹In]-DOTA-anti-IGF-1R conjugate (the “no cold” regimen) and subsequently patients received the same 5 mCi dose of the imaging agent [¹¹¹In]-DOTA-anti-IGF-1R conjugate and either 0.5 or 1.5 mg/kg of non-radioactive IGF-1R antibody AVE1642 (“cold antibody” dosing regimen) followed by imaging and pharmacokinetic assessment. Four patients had sufficient data with and without pre-administration of cold antibody to estimate radiation doses of [²²⁵Ac]-DOTA-anti-IGF-1R conjugate to the whole body, liver, spleen, red bone marrow, and kidneys. One patient was unable to complete all imaging time points needed for dosimetry. All five patients were evaluated for the comparison of lesion uptake where up to three lesions and backgrounds consisting of liver, muscle, red bone marrow, and total body were sampled and compared in each patient. All five patients were also evaluated for the determination of [¹¹¹In]-DOTA-anti-IGF-1R conjugate plasma pharmacokinetics as measured by total radioactivity.

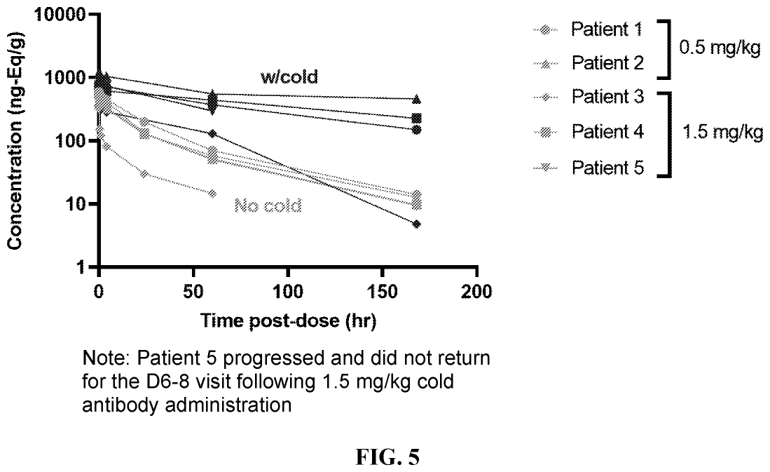

It was observed that the pre-administration of cold antibody prior to the [¹¹¹In]-DOTA-anti-IGF-1R conjugate dosing regimen had a significant effect on the plasma pharmacokinetics of [¹¹¹n]-DOTA-anti-IGF-1R conjugate in all five patients. See FIG. 5. In each patient, the clearance of the [¹¹¹In]-DOTA-anti-IGF-1R conjugate was markedly reduced in the cold antibody dosing regimen at both the 0.5 and 1.5 mg/kg dose levels. The pharmacokinetics of [¹¹¹In]-DOTA-anti-IGF-1R conjugate in each dose level of IGF-1R antibody AVE1642 (i.e., 0.5 or 1.5 mg/kg) were comparable, suggesting that doses equal to or greater than 0.5 mg/kg are sufficient to saturate the endogenous antigen sink and achieve linear pharmacokinetics. On average for the five evaluable patients, the pre-administration of IGF-1R antibody AVE1642 to the dosing regimen increased the total exposure (AUC_{inf}) of [¹¹¹In]-DOTA-anti-IGF-1R conjugate by 6-fold relative to [¹¹¹In]-DOTA-anti-IGF-1R conjugate only. See Table 2 below.

TABLE 2
Pharmacokinetics of IGF-1R-targeting radioimmunoconjugates
with/without cold IGF-1R antibody pre-administration
Pharmacokinetic Parameters

| | | [¹¹¹In]-DOTA-anti-IGF-1R alone | | | [¹¹¹In]-DOTA-anti-IGF-1R + AVE1642 | | |
|---|---|---|---|---|---|---|---|
| Patient | AVE1642 | Cₘₐₓ | AUC_{inf} | T_{1/2} | Cₘₐₓ | AUC_{inf} | T_{1/2} |
| ID | dose level | (ng-Eq/mL) | (ng-Eq*hr/mL) | (hr) | (ng-Eq/mL) | (ng-Eq*hr/mL) | (hr) |
| 1 | 0.5 mg/kg | 595 | 18600 | 39.4 | 806 | 61300 | 69.3 |
| 2 | 0.5 mg/kg | 535 | 13900 | 44.7 | 670 | 103000 | 121 |
| 3 | 1.5 mg/kg | 323 | 2840 | 20.5 | 356 | 20300 | 28.1 |
| 4 | 1.5 mg/kg | 568 | 13400 | 39.6 | 733 | 106000 | 115 |
| 5 | 1.5 mg/kg | 754 | 14500 | 39.6 | 896 | 48400 | 38.7 |

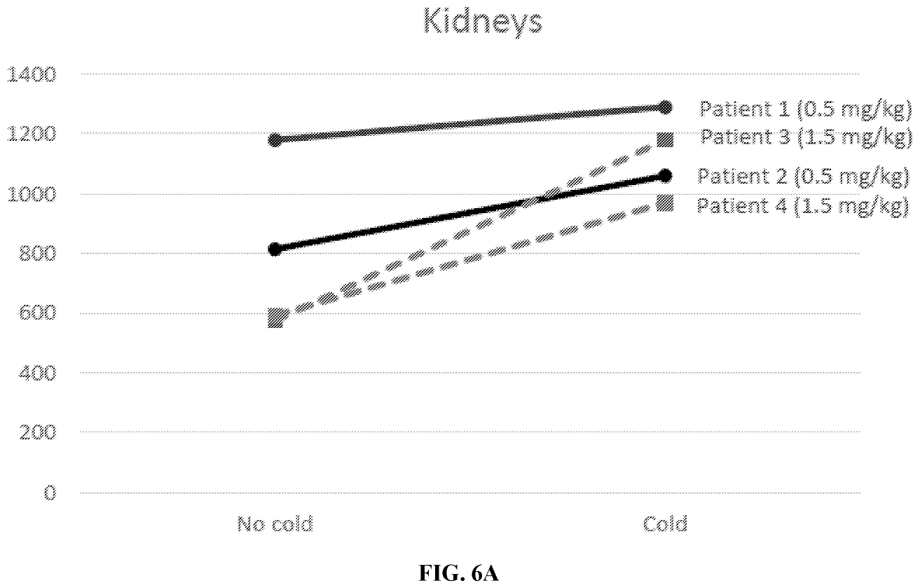

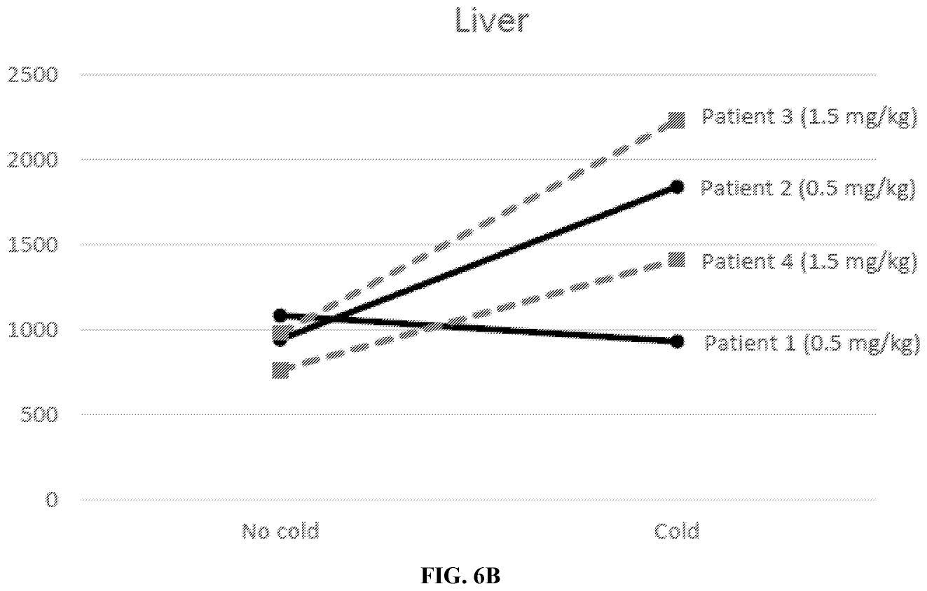

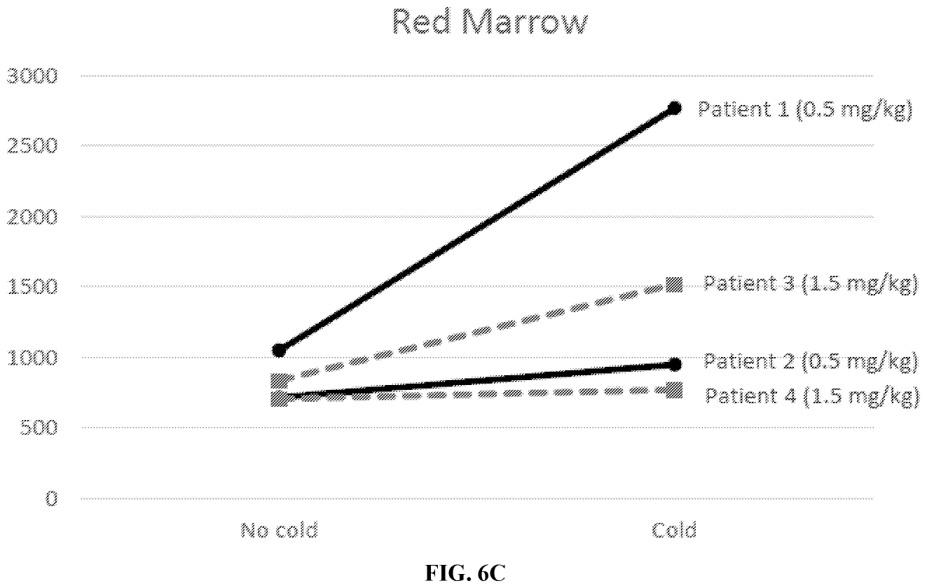

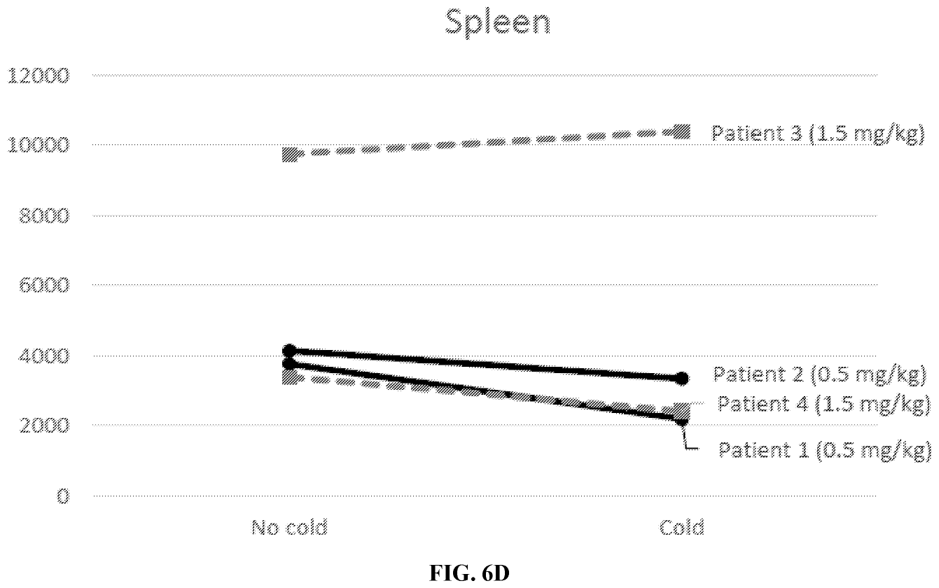

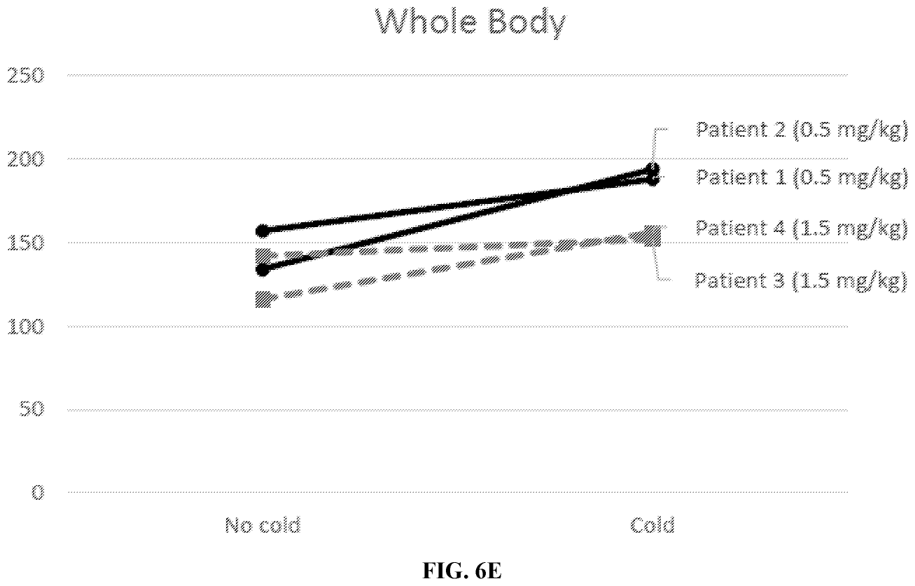

Among the five patients, imaging study shows that, at the cold antibody dose level of 0.5 mg/kg, radiation dose to the whole body, kidneys, and red marrow increased with the addition of cold antibody, while radiation dose to the liver was decreased in one patient, stayed about the same in another patient. Radiation dose to spleen decreased in both patients. At the cold antibody dose level of 1.5 mg/kg, radiation dose to the whole body, kidneys, liver, and red marrow increased. There was a mixed result in the spleen at this dose level with one patient showing a slight increase while the other showed a decrease in radiation dose with the addition of cold antibody. See FIGS. 6A-6E.

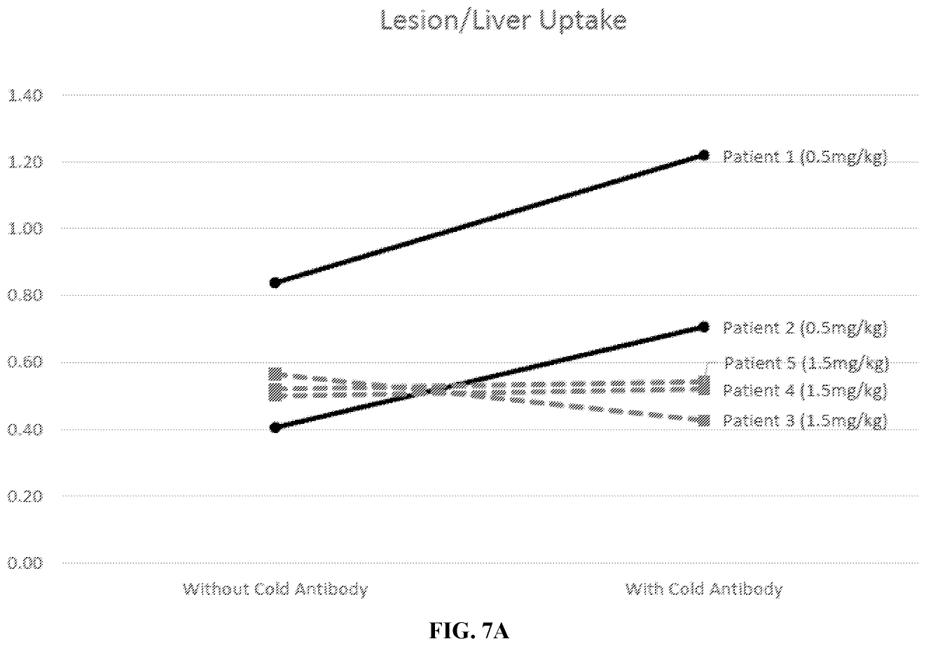

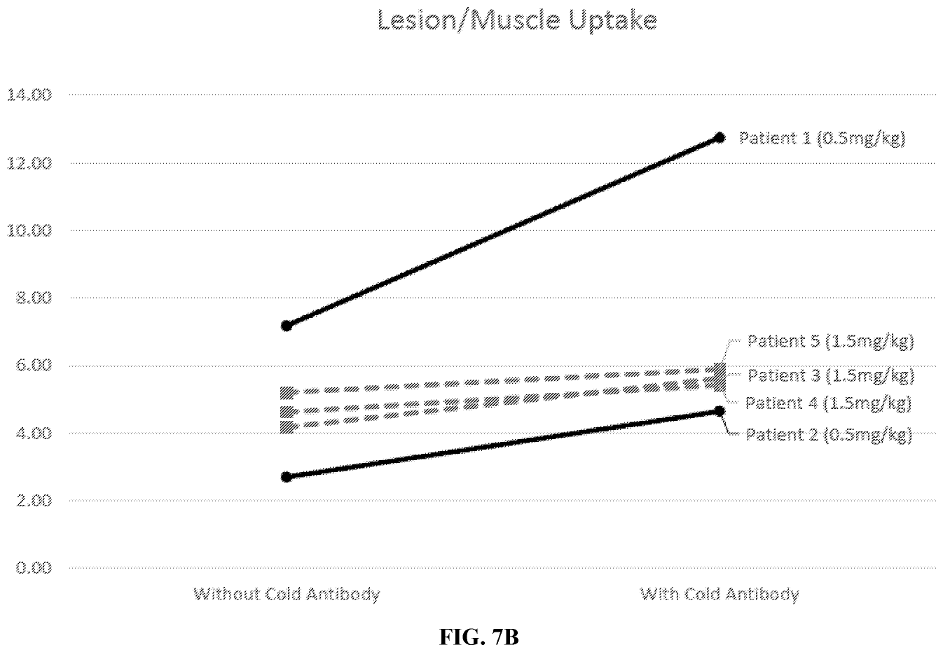

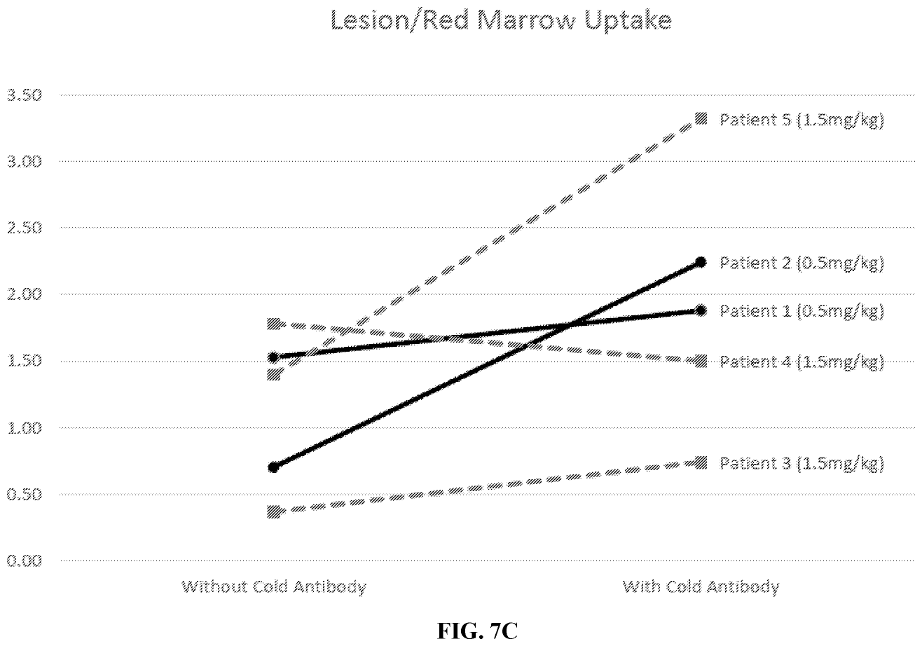

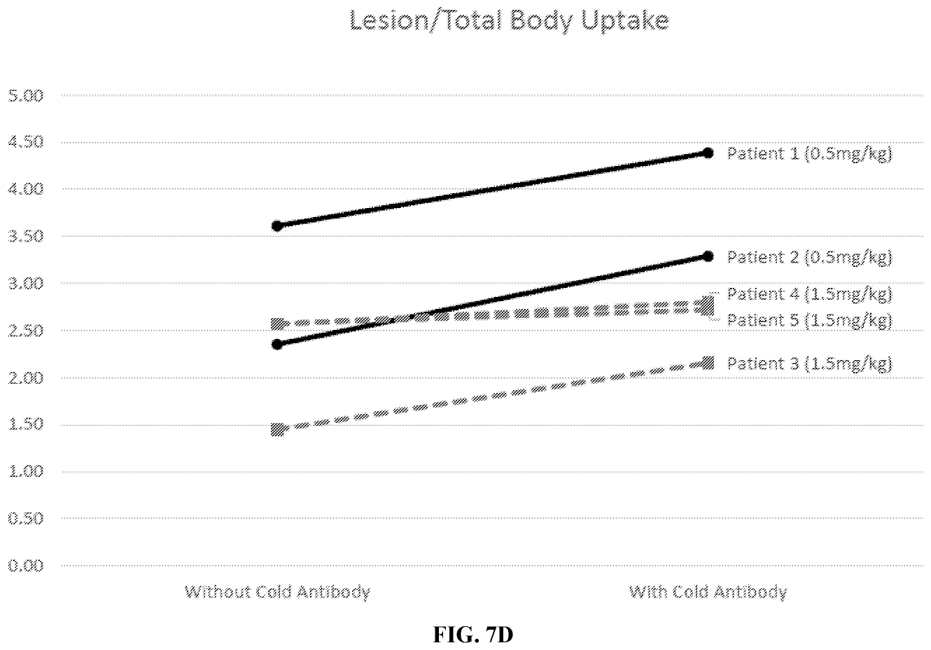

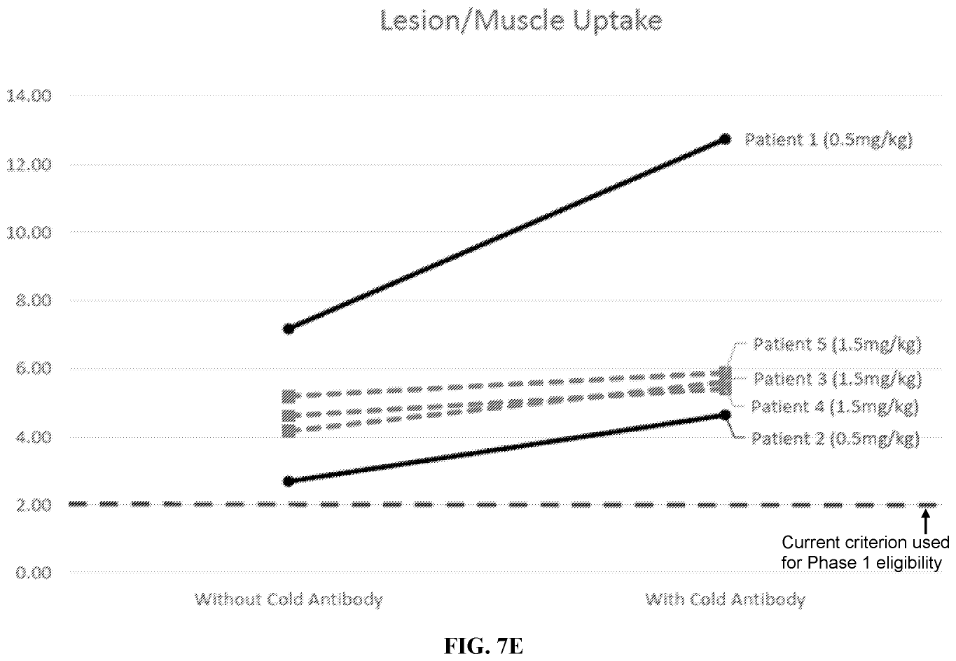

Further, ratios of tumor uptake relative to background increased for each patient at the cold antibody dose level of 0.5 mg/kg and 1.5 mg/kg. It was noted that the relative gain in tumor uptake was greater in patients treated with 0.5 mg/kg (n=2) compared to those treated in the 1.5 mg/kg group (n=3). This study demonstrated that pre-administration of cold IGF-1R antibody increased tumor lesion uptake of radioimmunoconjugate targeting IGF-1R in all patients. See FIGS. 7A-7E.

### Example 6. Efficacy of IGF-1R-Targeting Radioimmunoconjugates Involving Cold IGF-1R Antibody Pre-Administration

The above findings indicate that the pre-administration of cold IGF-1R antibody AVE1642 at 0.5 mg/kg prior to treatment with [²²⁵Ac]-DOTA-anti-IGF-1R conjugate shall improve uptake of the compound leading to a greater deposition of radiation to the tumor relative to the normal tissues, thereby likely increasing efficacy and decreasing off-target toxicities.

In addition, based on the imaging and pharmacokinetic data above, the predicted therapeutic efficacious range of [²²⁵Ac]-DOTA-anti-IGF-1R conjugate can be extrapolated from non-clinical mouse xenograft efficacy data as below: without cold antibody, the [²²⁵Ac]-DOTA-anti-IGF-1R conjugate is expected to provide radioactivity in the range of 80-160 kBq/kg cumulative exposure, while with pre-administration of cold IGF-1R antibody, the [²²⁵Ac]-DOTA-anti-IGF-1R conjugate is expected to provide radioactivity in 35-70 kBq/kg cumulative exposure. Accordingly, when pre-administered with cold IGF-1R antibody (AVE1642) at 0.5 mg/kg, [²²⁵Ac]-DOTA-anti-IGF-1R conjugate can be administered at a dosage of 15 kBq/kg, 20 kBq/kg, 25 kBq/kg, 30 kBq/kg, or 35 kBq/kg to achieve therapeutic efficacy.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

What is claimed is:

1. A method of treating a patient having cancer, wherein the method comprises administering to a patient in need thereof an effective amount of a radioimmunoconjugate or a pharmaceutically acceptable salt thereof,
wherein the radioimmunoconjugate comprises the following structure:
A-L-B Formula I
wherein
A is a metal complex of a chelating moiety,
B is an insulin-like growth factor 1 receptor (IGF-1R) targeting moiety, and
L is a linker,
wherein the patient is being co-administered a cold IGF-1R-targeting molecule.

2. The method of claim 1, wherein B is an antibody or an antigen-binding fragment thereof that is capable of binding to IGF-1R and the cold IGF-1R-targeting molecule is a cold IGF-1R antibody or IGF-1R binding fragment thereof.

3. The method of claim 2, wherein the cold IGF-1R antibody is pre-administered to the patient.

4. The method of claim 3, wherein the cold IGF-1R antibody is pre-administered at a dosage of 0.1 mg/kg to 10 mg/kg based on the body weight of the patient.

5. The method of claim 4, wherein the cold IGF-1R antibody is pre-administered at a dosage of 0.5 mg/kg to 3 mg/kg based on the body weight of the patient.

6. The method of any one of claims 1-5, wherein the radioimmunoconjugate is administered at a dosage of 10 kBq to 100 kBq/kg of body weight of said patient.

7. The method of any one of claims 1-6, wherein the radioimmunoconjugate is administered at a dosage of 15 kBq to 80 kBq/kg of body weight of said patient.

8. The method of any one of claims 1-7, wherein the radioimmunoconjugate is administered with a cumulative exposure of 20 kBq to 300 kBq/kg of body weight of said patient.

9. The method of any one of claims 1-8, wherein the radioimmunoconjugate is administered with a cumulative exposure of 35 kBq to 70 kBq/kg of body weight of said patient.

10. The method of claim 1, wherein L has the structure L¹-(L²)ₙ, as shown within Formula II:
A-L¹-(L²)ₙ-B Formula II
wherein
A is a metal complex of a chelating moiety;
B is an antibody or an antigen-binding fragment thereof that is capable of binding to IGF-1R;
L¹ is a bond, C═O, C═S, optionally substituted C₁-C₆ alkyl, optionally substituted C₁-C₆ heteroalkyl, or optionally substituted aryl or heteroaryl;
n is an integer between 1 and 5 (inclusive); and
each L², independently, has the structure:
—X¹-L³-Z¹—
wherein
X¹ is —C(O)NR¹—*, —NR¹C(O)—*, —C(S)NR¹—*, —NR¹C(S)—*, —OC(O)NR¹—*, —NR¹C(O)O—*, —NR¹C(O)NR¹—, —CH₂-Ph-C(O)NR¹—*, —NR¹C(O)-Ph-CH₂—*, —CH₂-Ph-NH—C(S)NR¹—*, —NR¹C(S)—NH-Ph-CH₂—*, —O—, or —NR¹—, wherein “*” indicates the attachment point to L³, and each R¹ is independently hydrogen, optionally substituted C₁-C₆ alkyl, optionally substituted C₁-C₆ heteroalkyl, or optionally substituted aryl or heteroaryl;
L³ is optionally substituted C₁-C₅₀ alkyl or optionally substituted C₁-C₅₀ heteroalkyl; and
Z¹ is —CH₂—, —C(O)—, —C(S)—, —OC(O)—#, —C(O)O-#, —NR²C(O)—#, —C(O)NR²-#, or —NR²—, wherein “#” indicates the attachment point to B, and each R² is independently hydrogen, optionally substituted C₁-C₆ alkyl, optionally substituted C₁-C₆ heteroalkyl, optionally substituted aryl, or optionally substituted heteroaryl.

11. The method of claim 10, wherein L³ comprises (CH₂CH₂O)₂₋₂₀ or (CH₂CH₂O)₂₋₂₀—C₁-C₆ alkyl.

12. The method of any one of claims 1-11, wherein the chelating moiety is selected from the group consisting of DOTA (1,4,7,10-tetraazacyclododecane-1,4,7,10-tetraacetic acid), DOTMA (1R,4R,7R,10R)-α, α′, α″, α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DO3AM-acetic acid (2-(4,7,10-tris(2-amino-2-oxoethyl)-1,4,7,10-tetraazacyclododecan-1-yl)acetic acid), DOTP (1,4,7,10-tetraazacyclododecane-1,4,7,10-tetra(methylene phosphonic acid)), DOTA-4AMP (1,4,7,10-tetraazacyclododecane-1,4,7,10-tetrakis(acetamido-methylenephosphonic acid), NOTA (1,4,7-triazacyclononane-1,4,7-triacetic acid), and HP-DO3A (10-(2-hydroxypropyl)-1,4,7-tetraazacyclododecane-1,4,7-triacetic acid).

13. The method of any one of claims 1-12, wherein the metal complex comprises a radionuclide selected from the group consisting of ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁸⁹Zr, ⁹⁰Y, ⁹⁷Ru, ⁹⁹Tc, ^{99m}Tc, ¹⁰⁵Rh, ¹⁰⁹Pd, ¹¹¹In, ^{117m}Sn, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

14. The method of any one of claims 1-13, wherein L has the structure -L¹-(L²)ₙ-, as shown within Formula II:
A-L¹-(L²)ₙ-B Formula II
wherein:
A is a metal complex of DOTA;
B is an antibody or an antigen-binding fragment thereof that is capable of binding to IGF-1R;
L¹ is a bond or C₁-C₆ alkyl;
n is 1; and
L² has the structure:
—X¹-L³-Z¹—
wherein:
X¹ is —C(O)NR¹—*, “*” indicating the attachment point to L³, and R¹ is H or C₁-C₆ alkyl;
L³ is (CH₂CH₂O)ₘ(CH₂)_{w}, and m and w are independently an integral between 0 and 10 (inclusive) and at least one of m and w is not 0; and
Z¹ is —C(O)—.

15. The method of claim 1, wherein A-L- is a metal complex of a moiety selected from the group consisting of:

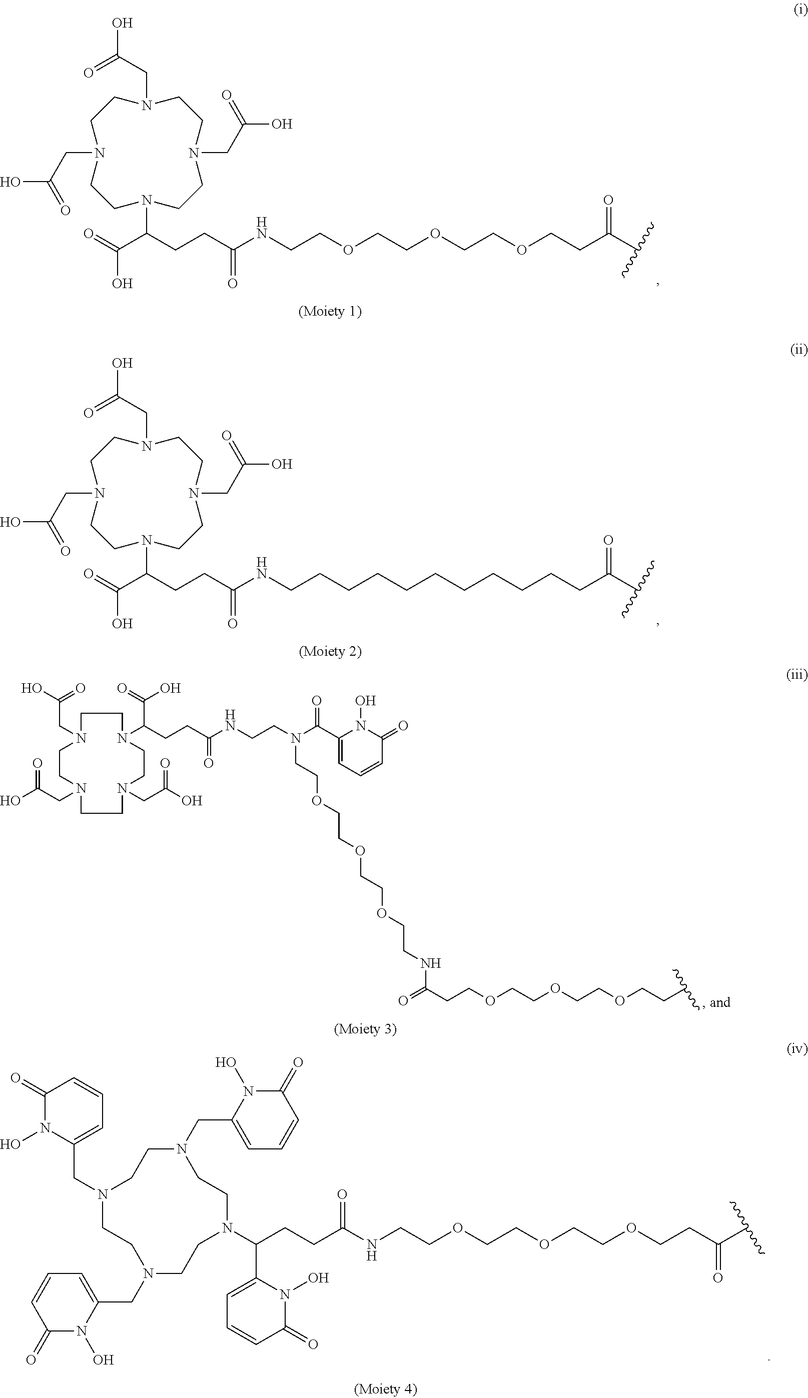 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O ; *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

16. The method of claim 15, wherein A-L- is a metal complex of Moiety 1:

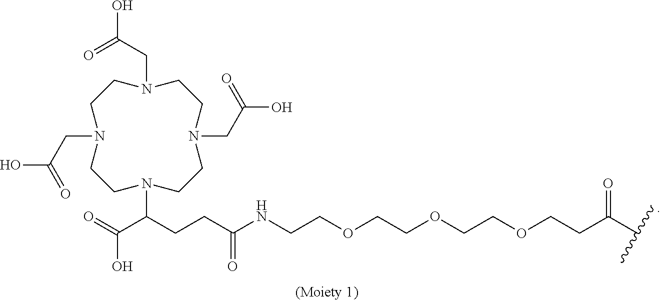 [SMILES: *C(=O)CCOCCOCCOCCNC(=O)CCC(C(=O)O)N1CCN(CC(=O)O)CCN(CC(=O)O)CCN(CC(=O)O)CC1]

17. The method of any one of claims 1-16, wherein the radioimmunoconjugate comprises the following structure: 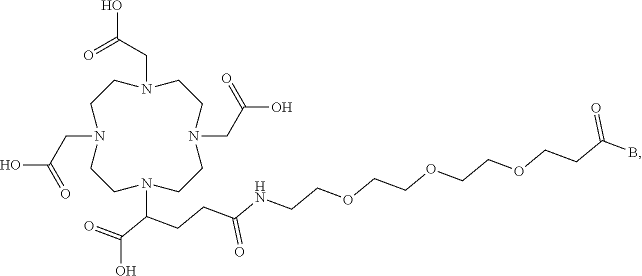
wherein B is an antibody or an antigen-binding fragment thereof that is capable of binding to IGF-1R.

18. The method of any one of claims 1-17, wherein the metal complex comprises an alpha emitter.

19. The method of claim 18, wherein the alpha emitter is selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (149Tb), or a progeny thereof.

20. The method of claim 19, wherein the metal complex comprises ²²⁵Ac or a progeny thereof.

21. The method of any one of claims 1-20, wherein the radioimmunoconjugate comprises the following structure: 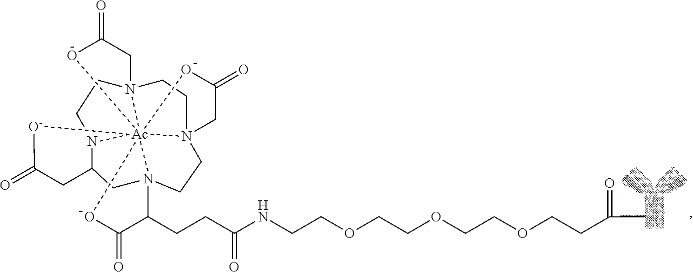
wherein [symbol] is AVE1642.

22. The method of any one of claims 1-21, wherein the cold IGF-1R-targeting molecule is AVE1642 or an IGF-1R-binding fragment thereof.

23. The method of any one of claims 1-22, wherein the cancer is a solid tumor cancer.

24. The method of claim 23, wherein the solid tumor cancer is selected from the group consisting of adrenocortical carcinoma, bladder cancer, breast cancer, cervical cancer, colorectal cancer, endometrial adenocarcinoma, Ewing's sarcoma, gallbladder carcinoma, glioma, head and neck cancer, liver cancer, lung cancer, neuroblastoma, neuroendocrine cancer, ovarian cancer, pancreatic cancer, prostate cancer, renal cell carcinoma, salivary adenoid cystic cancer, spermatocytic seminoma, and uveal melanoma.
